(19) United States
(12) Patent Application Publication
(10) Pub. No.: US 2023/0190934 A1
(43) Pub. Date: Jun. 22, 2023

(54) METHODS AND COMPOSITIONS FOR TREATING A TUMOR

(71) Applicant: Rakuten Medical, Inc., San Diego, CA (US)

(72) Inventors: Miguel GARCIA-GUZMAN, San Diego, CA (US); C. Daniel DE MAGALHAES FILHO, La Jolla, CA (US)

(73) Assignee: Rakuten Medical, Inc., San Diego, CA (US)

(21) Appl. No.: 17/924,979

(22) PCT Filed: May 13, 2021
(86) PCT No.: PCT/US2021/032339

§ 371 Date: Nov. 11, 2022

Related U.S. Application Data
(60) Provisional application No. 63/034,250, filed on Jun. 3, 2020.
(60) Provisional application No. 63/025,085, filed on May 14, 2020.

(51) Int. Cl. A61K 41/00; A61K 47/68

(52) U.S. Cl. CPC A61K 41/0071; A61K 47/6849; A61K 47/6803

(57) ABSTRACT

Provided are methods and uses for treating a cancer or a tumor. In some aspects, the provided methods and uses involve contacting a sample, e.g., containing tumor cells, with a phthalocyanine dye conjugated to a targeting molecule that binds a protein on tumor cell, and illuminating the sample with a wavelength of light suitable for the activation of the phthalocyanine dye. In some aspects, the methods and uses also involve administering the illuminated sample to a subject, such as a subject having a cancer or a tumor. The methods and uses described herein provide for stimulation of the anti-cancer immune response in the subject and the reduction of growth and/or elimination of cancers, tumors and tumor cells in the subject. Also provided are compositions and combinations for use in the provided methods.

### RELATED APPLICATIONS

This application claims priority from U.S. Provisional Application No. 63/025,085, filed May 14, 2020, entitled “METHODS AND COMPOSITIONS FOR TREATING A TUMOR” and U.S. Provisional Application No. 63/034,250, filed Jun. 3, 2020, entitled “METHODS AND COMPOSITIONS FOR TREATING A TUMOR,” the contents of which are incorporated by reference in their entirety.

### FIELD

The present disclosure relates to methods and uses for treating a cancer or a tumor. In some aspects, the provided methods and uses involve contacting a sample, e.g., containing tumor cells, with a phthalocyanine dye conjugated to a targeting molecule that binds a protein on tumor cell, and illuminating the sample with a wavelength of light suitable for the activation of the phthalocyanine dye. In some aspects, the methods and uses also involve administering the illuminated sample to a subject, such as a subject having a cancer or a tumor. The methods and uses described herein provide for stimulation of the anti-cancer immune response in the subject and the reduction of growth and/or elimination of cancers, tumors and tumor cells in the subject. The disclosure also relates to compositions and combinations for use in the provided methods.

### BACKGROUND

Every year many therapeutics for treating cancer are developed, including immune checkpoint inhibitors, small molecule targeted therapies, and other anticancer therapeutics. However, some patients are not responsive or not fully responsive to those therapeutics, leading to disease progression and cancer-related deaths. Novel compositions and methods are urgently needed to address these clinical challenges. Provided are methods, uses and compositions that meet such needs.

### SUMMARY

Provided herein are methods of treating a tumor or a lesion, including primary tumors or lesions as well as metastatic tumor cells. Also provided herein are methods of preventing tumor growth, such as prophylactic treatment of a tumor or a lesion.

Provided in some embodiments is a method of treating a tumor or a lesion that includes contacting a sample of cells with a conjugate comprising a phthalocyanine dye, such as a silicon phthalocyanine dye, linked to a targeting molecule; after contacting with the conjugate, illuminating the sample at a wavelength of at or about 600 nm to at or about 850 nm to obtain an illuminated sample; and administering the illuminated sample to a first subject.

Provided herein are methods of treating a tumor or a lesion that involve administering to a first subject a composition comprising an illuminated sample, wherein the illuminated sample comprises tumor cells that have been treated ex vivo with photoimmunotherapy. In some of any embodiments, the photoimmunotherapy involves contacting a sample of tumor cells ex vivo with a conjugate comprising a phthalocyanine dye, such as a silicon phthalocyanine dye, linked to a targeting molecule; and after contacting with the conjugate, illuminating the sample at a wavelength of at or about 600 nm to at or about 850 nm to obtain the illuminated sample. In some of any embodiments, the tumor cells or a portion thereof within the illuminated sample exhibit one or more markers of immunogenic cell death (ICD).

Also provided are methods of treating a tumor or a lesion that involve contacting a sample of tumor cells ex vivo with a conjugate comprising a phthalocyanine dye, such as a silicon phthalocyanine dye, linked to a targeting molecule; after contacting with the conjugate, illuminating the sample at a wavelength of at or about 600 nm to at or about 850 nm to obtain an illuminated sample; administering a composition comprising the illuminated sample to a first subject.

In some of any embodiments, the tumor cells or a portion thereof within the illuminated sample exhibit one or more markers of immunogenic cell death (ICD). In some of any embodiments, the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

In some of any embodiments, the sample comprises tumor cells derived or obtained from the first subject. the sample comprises tumor cells derived or obtained from a second subject. In some of any embodiments, the sample of cells is obtained from a biopsy of the first subject. In some of any embodiments, the sample of cells is obtained from a second subject. In some of any embodiments, the sample of cells is obtained from a plurality of subjects.

In some embodiments, the sample comprises tumor cells that have been grown or cultivated in vitro prior to contacting with the conjugate. In some of any embodiments, the sample is grown or cultivated in vitro prior to contacting with the conjugate. In some of any embodiments, the sample is grown or cultivated into an organoid prior to contacting with the conjugate. In some of any embodiments, the sample comprises tumor cells that have been grown or cultivated into an organoid prior to contacting with the conjugate.

In some of any of the provided embodiments, the illuminated sample is administered by injection or by infusion to the first subject. In some of any embodiments, the illuminated sample is administered by implantation into the first subject.

In some of any embodiments, the targeting molecule comprises an antibody or an antigen binding fragment thereof. In some of any embodiments, the targeting molecule binds to a cell surface molecule. In some of any embodiments, the cell surface molecule is present on a tumor cell or a cell in the tumor microenvironment. In some of any embodiments, the sample of cells comprises the tumor cell or the cell in the tumor microenvironment. In some of any embodiments, the cell surface molecule is present on a first tumor cell or a first cell in the tumor microenvironment. In some of any embodiments, the sample comprises the first tumor cell or the first cell in the tumor microenvironment.

In some of any embodiments, the cell surface molecule is selected from the group consisting of HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, a ganglioside, VEGF receptor (VEGFR), VEGFR2, VEGF-A, integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, SK-1antigen, Bcr-abl, RET, MET, TRKB, TIE2, ALK, ROS, EML4-ALK, ROS1, BRAFV600E, SRC, c-KIT, PDGFR, mTOR, TSC1, TSC2, BTK, KIT, BRCA, CDK 4/6, JAK1, JAK2, BRAF, FLT-3, MEK1, MEK2, and SMO.

In some of any of the provided embodiments, the phthalocyanine dye is a silicon phthalocyanine dye. In some of any embodiments, the silicon phthalocyanine dye is IR700.

In some of any embodiments, the targeting molecule is selected from the group consisting of cetuximab, panitumumab, zalutumumab, nimotuzumab, Tositumomab (Bexxar®), Rituximab (Rituxan, MabThera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and MNRP1685A and any antigen-binding fragment thereof.

In some of any embodiments, the method further includes administering a second treatment to the first subject, wherein the second treatment includes: administering to the first subject a second conjugate comprising a second phthalocyanine dye linked to a second targeting molecule, and, after administering the second conjugate, illuminating a tumor or a lesion in the first subject at a wavelength of at or about 600 nm to at or about 850 nm and at a dose of from at or about 25 J/cm² to at or about 400 J/cm² or from at or about 2 J/cm fiber length to at or about 500 J/cm fiber length. In some of such embodiments, the second treatment is administered subsequent to the administration of the illuminated sample to the first subject. In some of any embodiments, the second treatment is administered prior to the administration of the illuminated sample to the first subject. In some of any embodiments, the second treatment is administered prior to and subsequent to the administration of the illuminated sample to the first subject.

In any of the provided embodiments, the illuminated sample can be administered in combination with an immune modulatory agent. In some embodiments, the composition is administered in combination with an immune modulatory agent. In some of any embodiments, the immune modulatory agent administered prior to, concurrent with and/or subsequent to the illuminated sample. In some of any embodiments the immune modulatory agent comprises an adjuvant, an immune checkpoint inhibitor, a cytokine or any combination thereof.

In some of any of the provided embodiments, the sample of cells can be obtained from a tumor. In some of any embodiments, the sample of cells comprises tumor cells. In some of any embodiments, the tumor cells are obtained from multiple tumor sources.

In some of any embodiments, the first subject has been diagnosed as having, or is suspected of having, a type of cancer. In some of any of the provided embodiments, the first subject has, has been diagnosed as having, or is suspected of having a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.

In some of any of the provided embodiments, the sample of cells comprises cells from a tumor or a lesion associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof. In some of any embodiments, the sample comprises tumor cells that are derived from the same or similar type of cancer that the first subject has been diagnosed as having, or is suspected of having.

In some provided methods, the illuminated sample comprises less than 100% cell death prior to administration to the first subject. In some provided methods, the illuminated sample comprises at least 30%-70% cell death prior to administration to the first subject. In some of any of the provided embodiments, the sample is treated to prevent cell growth or cell expansion prior to administration to the first subject. In some of any embodiments, the sample is treated with irradiation prior to administration. In some of any embodiments, irradiation comprises gamma irradiation. In some of any embodiments, irradiation is gamma irradiation.

In some of any embodiments, administering the composition results in a stimulation of an anti-cancer immune response in the first subject. In some of any embodiments, administering the composition results in a reduction of growth, a reduction in size, a reduction in volume, or elimination of a tumor, a lesion or a metastasis in the first subject.

Provided herein are pharmaceutical compositions comprising photoimmunotherapy-treated tumor cells. In some of any embodiments, the pharmaceutical composition comprises at least one pharmaceutically acceptable excipient. Provided herein are pharmaceutical compositions comprising photoimmunotherapy-treated tumor cells formulated with at least one pharmaceutically acceptable excipient. Also provided herein are any of the provided pharmaceutical compositions for use in treating a tumor or a lesion. Also provided are uses of any of the provided pharmaceutical compositions, in treating a tumor or a lesion. Also provided are uses of any of the provided pharmaceutical composition in the manufacture of a medicament for treating a tumor or a lesion.

In some of any embodiments, the tumor cells or a portion thereof in the pharmaceutical composition exhibit one or more markers of immunogenic cell death (ICD). In some of any embodiments, the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

In some of any embodiments, the pharmaceutical composition comprises tumor cells derived from a single subject. In some of any embodiments, the pharmaceutical composition comprises tumor cells derived from more than one subject.

In some of any embodiments, the pharmaceutical composition comprises tumor cells derived from a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.

### BRIEF DESCRIPTION OF THE DRAWINGS

FIG. 1 shows the level of released Annexin A1 (pg/mL) from A431, BxPC3 and FaDu cells incubated with an antibody-IR700 conjugate, followed by light illumination (after PIT; white bars) or without light illumination (no PIT; black bars).

FIG. 2 shows the level of released adenosine triphosphate (ATP, x 10⁻⁷ M) from 4T1-EpCam, A431, BxPC3, CT26-EphA2 and LL/2-EphA2 cells incubated with an antibody-IR700 conjugate, followed by light illumination (after PIT; white bars) or without light illumination (no PIT; black bars).

FIG. 3A shows the level of ATP released (10⁻⁷ M) from 4T1-EpCam, A431, BxPC3, CT26-EphA2, FaDu, and LL/2-EphA2 cells 26 hours after PIT (Conjugate+light; white bars), or untreated cells (no treatment; black bars).

FIG. 3B shows a time course of ATP released (10⁻⁷ M; solid line) and percent cell death (dashed line) in response to PIT.

FIGS. 4A and 4B show the release of interferon α2 (IFN-α2) and interferon β (IFN-β) release (pg/mL), respectively, from A431, BxPC3, and FaDu cells incubated with antibody-IR700 conjugate and illuminated (Conjugate+light; white bars) or untreated (No treatment; black bars).

FIG. 5 shows the release of High-Mobility Group Protein B1 (HMGB1) release (ng/mL), respectively, from A431, and FaDu cells incubated with antibody-IR700 conjugate with (white bars) or without (black bars) illumination.

FIGS. 6A and 6B show the increase in heat shock protein and calreticulin surface expression on A431 cells and FaDu cells, respectively, that were incubated with antibody-IR700 conjugate with (white bars) or without (black bars) illumination.

FIG. 7A shows the fold change in expression of differentiation 86 (CD86) and major histocompatibility complex II (MHCII) markers on dendritic cells (DCs) following exposure to the supernatant of tumor cells incubated with antibody-IR700 conjugate with (white bars) and without (black bars) illumination.

FIG. 7B shows dendritic cell production of pro-inflammatory cytokines tumor necrosis factor (TNF), IFN-γ-Inducible Protein 10 (IP-10), MIP-1α (Macrophage Inflammatory Protein-1 alpha), MIP-1β (Macrophage Inflammatory Protein-1 beta), interleukin-1 beta (IL-1β) and interleukin-8 (IL-8) following exposure to the supernatant of tumor cells incubated with antibody-IR700 conjugate with (white bars) and without (black bars) illumination.

FIG. 8A shows the average tumor volumes over time of mice that were treated with anti-EphA2 conjugate alone (open circles), anti-EphA2-PIT (closed circles), and anti-EphA2-PIT preceded by anti-CD40L treatment (closed triangles).

FIG. 8B shows the tumor volumes over time of naïve mice inoculated with tumor cells and complete responder (CR) mice following anti-tumor PIT re-challenged with tumor cells.

FIG. 8C shows the average tumor volumes over time of mice that were treated with anti-EphA2 conjugate alone (open circles), anti-EphA2-PIT (closed circles), and anti-EphA2-PIT together with anti-CD40L treatment (closed triangles).

FIG. 8D shows the average tumor volumes over time of mice that were treated with anti-EphA2 conjugate alone (open circles), anti-EphA2-PIT (closed circles), anti-PD1 alone (open squares), anti-PD1 and anti-EphA2-PIT (closed squares), and anti-PD1 and anti-EphA2-PIT with depletion of CD8⁺ T-cells by anti-CD8 administration (closed triangles).

FIG. 9 shows a schematic depicting the experimental steps and tumor volume changes over time of an implanted tumor (between approximately 5 to 24 days after tumor implantation) in mice that had been administered CT26-EphA2 cells incubated with cisplatin (cisplatin group) or CT26-EphA2 cells incubated with an exemplary antibody-IR700 conjugate and treated with light illumination (PIT group).

FIGS. 10A-10C show the tumor volumes over time of individual mice that were vaccinated with PIT-treated tumor cells undergoing cell death (FIG. 10A), 100% expired PIT-treated tumor cells (FIG. 10B), or saline only (FIG. 10C).

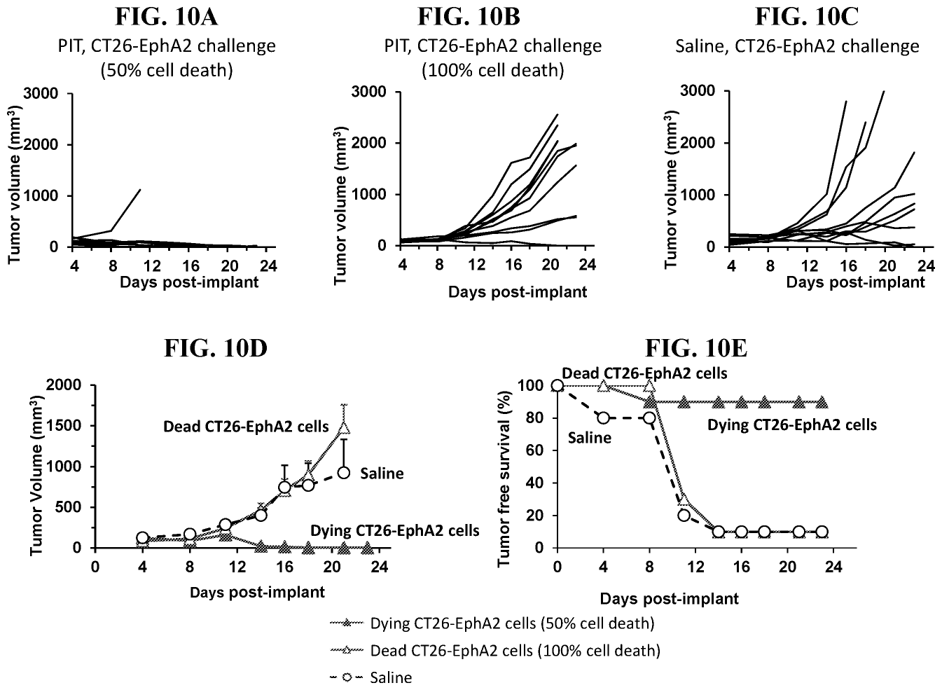

The average tumor and tumor free-survival, of animals vaccinated with dying PIT-treated tumor cells (closed triangles), dead PIT-treated tumor cells (open triangles) or saline control animals (open circles) are plotted in FIG. 10D and FIG. 10E, respectively.

### DETAILED DESCRIPTION

Provided herein are composition and methods for use in cancer treatment. In some aspects, provided are methods and uses for treating a cancer, a tumor or a lesion. In some embodiments, the compositions and methods provide for vaccines for stimulating, enhancing, augmenting or boosting an immune response, such as an anti-cancer immune response, in a subject having cancer or a population of tumor cells, or a subject suspected of having a cancerous lesion. In some embodiments, the compositions and methods generate immunity, such as anti-cancer immunity, or stimulate, enhance, augment or boost pre-existing immunity. In some embodiments, the compositions and methods are used to stimulate, enhance, augment or boost the immunogenic response to a therapeutic agent. The methods and uses described herein provide for stimulation of the anti-cancer immune response in a subject and the reduction of growth and/or elimination of cancers, tumors and tumor cells in the subject.

The methods and uses provided herein and compositions for use therewith include an ex vivo treatment of a sample, such as a sample containing cells, for example tumor cells, followed by the introduction, for example by administration, of the treated sample into a subject. In some embodiments, the provided methods and uses involve obtaining a sample containing a cancer or a tumor; contacting the sample with a phthalocyanine dye conjugated to a targeting molecule that binds a protein on tumor cell, for example, an IR700-antibody conjugate, and illuminating the sample with a wavelength of light suitable for the activation of the phthalocyanine dye. In some embodiments, the methods and uses also involve administering the illuminated sample (or a composition comprising the illuminated sample) to a subject, such as a subject having a cancer or a tumor. In some embodiments, the illuminated sample contains cells undergoing cell death. Also provided are compositions and combinations for use in the provided methods and uses.

In some aspects, the illuminated sample, such as an illuminated sample comprising tumor cells, are employed in the provided compositions, combinations, methods and uses. Uses include uses of the compositions in such methods, such as therapeutic methods, and treatments, such as a treatment regimen, and uses of such compositions in the preparation of a medicament, in order to carry out such therapeutic methods and treatments. Also provided are such compositions for use in treating a tumor, a lesion or a cancer. In some aspect, such uses include performing the methods or treatments as described herein, such as any therapeutic methods or treatment regimens. In some embodiments, the illuminated sample or compositions comprising the same, are prepared by a method involving photoimmunotherapy, for example, that involve contacting the sample with a conjugate comprising a phthalocyanine dye linked to a targeting molecule and illuminating the sample, for example ex vivo, with light.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Samples and Ex Vivo Illumination

The compositions and methods herein involve obtaining a sample, such as a biological sample, from a subject. In some aspects, the sample includes live cells. In some embodiments, the subject has one or more tumors and the live cells are taken from one or more of the tumors. In some embodiments, the subject is suspected of having a cancer, and a sample of live cells is taken of the tissue or organ or cell type suspected of harboring cancerous or pre-cancerous cells. In some embodiments, the sample is taken from blood, from bone marrow, or from a lymph node.

In some embodiments, the sample is collected from a subject having or suspected of having a cancer, such as a cancer selected from colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals. In some embodiments, the sample is collected from a subject having or suspected of having a blood cancer. Exemplary blood cancers include leukemia, lymphoma, and myeloma, such as multiple myeloma.

The sample includes one or more live cells. In some embodiments, the sample is a single cell type. In some embodiments, the sample is composed of multiple cell types. In some embodiments, the sample is a single cell. In some embodiments, the sample is composed of cells isolated from blood, such as by leukapheresis.

In some embodiments, an expanded sample is generated, for example by growing the sample containing live cells in vitro. In some embodiments, the sample is grown as a cell culture. In some embodiments, the sample is grown into one or more organoids. In some embodiments the sample or co-cultured sample is treated or manipulated to isolate or select for one or more cell types. In some embodiments, the sample of live cells is co-cultured with other cells. In some embodiments, the other cells used in co-culture are dendritic cells. The sample or co-cultured sample can be grown in vitro for a period of time, such as at least 1, 2, 3, 4, 5, days, for example, 1-5 days, 1-10 days, 1 week, 2 weeks, 3 weeks or more than 3 weeks. In some embodiments, the sample, e.g., containing cells, can be grown to a certain density or cell number. In some embodiments, the sample, e.g., containing cells, can be grown until they form a certain morphology such as a particular organoid morphology.

The sample or expanded sample (or a portion thereof) is treated with photoimmunotherapy to induce immunogenic cell death. In some embodiments, photoimmunotherapy is applied using a conjugate of a phthalocyanine dye linked to a targeting molecule that binds to a protein on a cell surface. In some embodiments, photoimmunotherapy is applied by contacting the sample or expanded sample, e.g., containing cells, with a conjugate of a phthalocyanine dye linked to a targeting molecule that binds to a protein on a cell surface, such as the surface of a cell present in the sample or the expanded sample. In some embodiments, the conjugate is a phthalocyanine dye linked to a targeting molecule, such as an antibody, an antigen-binding fragment thereof or an antibody-like molecule (such as a nanobody, affibody) that binds to a protein on the surface of a cell. In some aspects, exemplary phthalocyanine dye-targeting molecule conjugate employed in the methods provided herein, include any described herein, for example, in Section III. In some aspects, the sample is treated with photoimmunotherapy ex vivo or extracorporeally. For example, in some aspects, a sample containing tumor cells is obtained from a subject, taken outside of the subject's body, and is subjected to photoimmunotherapy.

In some embodiments, the conjugate binds to a cancer cell, such as a cancer cell present in the sample. In some embodiments, the phthalocyanine dye of the conjugate is a silicon phthalocyanine dye. In some embodiments, the phthalocyanine dye of the conjugate is IR700Dye (LiCor).

Following contacting with the phthalocyanine dye-targeting molecule conjugate or composition containing such conjugate, the sample or a portion thereof is illuminated (also referred herein as photoimmunotherapy (PIT) treatment). In some aspects, the sample is illuminated at a wavelength of 600-850 nm or a wavelength of 660-740 nm. In some embodiments, the illumination dosage is at least 1 J/cm², for example, at least 1 J/cm², at least 10 J/cm², at least 50 J/cm², at least 100 J/cm², for example, 1.0 to 500 J/cm². In some embodiments, the wavelength is 660-710 nm. In some embodiments, the illumination is performed at a wavelength of 690±50 nm or at a wavelength of 690±20 nm. In some embodiments, the illumination is performed more than once on the sample, such as 2, 3, 4, 5, 6, 7, 8, 9 or 10 separate illuminations. In some embodiments, the illumination occurs ex vivo, i.e., outside of the body of the subject from whom the sample was obtained. In some aspects, the illumination occurs ex vivo, after the sample has been expanded.

In some aspects, the contacting with the phthalocyanine dye-targeting molecule conjugate with the sample, e.g., containing cells, followed by illumination at a wavelength as described herein, results in immunogenic cell death of some or all of the cells in the sample. Immunogenic cell death is a specific type of cell death exhibited by necrotic cells and is characterized by increased presentation and release of immune stimulatory markers. Cells exhibiting ICD display membrane changes such as elevated surface expression of heat shock protein 90, and secretion of soluble, intracellular markers known as danger associated molecular patterns (DAMPs), such as ATP and high-mobility group-box protein (HMGB1) (Kromer et al. (2013) Annual Review of Immunology, 31:51-72). In some aspects, the illuminated sample in accordance with the methods described herein, contain cells that are undergoing or have undergone immunogenic cell death. In some aspects, the administration of the illuminated sample to a subject can stimulate, enhance, augment or boost an immunogenic response, such as an anti-cancer immune response, in the subject.

### II. Methods of Treating a Subject Using Illuminated Samples

In some aspects, the methods also involve administering (e.g., introducing) the illuminated sample to a subject having a disease, disorder or a lesion, such as a cancer or a tumor. In some aspects, after obtaining the sample from a subject, the sample is contacted with a phthalocyanine-targeting molecule conjugate, and illuminated (e.g., PIT treatment), as described above. In some aspects, the contact with a phthalocyanine-targeting molecule conjugate and the illumination occur extracorporeally. In some aspects, the illumination occurs ex vivo. In some embodiments, following PIT, the ex vivo illuminated (e.g., PIT-treated) sample is administered to a subject with a cancer or a tumor. In some embodiments, the illuminated sample is administered by injection. In some embodiments, the illuminated sample is administered by implantation into the subject. In some embodiments, the treatment can be a prophylactic treatment. In some embodiments, the treatment can be a therapeutic treatment.

In some embodiments, the PIT-treated cells are administered to a subject with a cancer or tumor about 15 minutes to about 24 hours following illumination of the ex vivo cells, such as 15 minutes to 12 hours, 0-8 hours, 2-6 hours after illumination of the ex vivo cells. In some examples, PIT-treated cells are administered 15 minutes, 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours after illumination ex vivo cells.

In some embodiments, the PIT-treated cells are administered to a subject once the cells have begun PIT-mediated cell death, such as PIT-mediated immunogenic cell death. In some embodiments, the PIT-treated cells are administered when the sample exhibits at least 10%, at least 20%, at least 30%, at least 40%, at least 50%, at least 60%, at least 70%, at least 80% or at least 90% cell death. In some examples, the PIT-treated cells are administered when less than 100% of the cells in the sample exhibit cell death. In particular examples, the PIT-treated cells are administered when the cells approximately 30%-70% viable or exhibit 30% to 70% cell death. In some embodiments, the cells are administered when the cells are approximately 50% viable. Percent cell death can be measured using a cell viability assay. Many of such assays are known and can be used to assess the viability of PIT-treated cells prior to administration.

In some embodiments, the PIT-treated cells are administered systemically, such as by intravenous administration. In some embodiments, the PIT-treated cells are administered intravenously in the form of a bolus dose or an infusion. In some embodiments, the PIT-treated cells are administered proximal to a tumor or lesion. In some embodiments, the PIT-treated cells are administered by intratumoral injection. In some embodiments, the PIT-treated cells are administered via the subcutaneous route. In some embodiments, the PIT-treated cells are administered via the intramuscular route.

In some embodiments, the PIT-treated cells are treated with an additional treatment to prevent cell expansion or cell division prior to administration to a subject. In some embodiments, the PIT-treated cells are gamma irradiated prior to administration to a subject. The additional treatment can be administered before or after the PIT treatment of the cells, prior to administration.

In some embodiments, a sample is obtained from a subject, subjected to PIT (e.g., contacted with phthalocyanine-targeting molecule conjugate and illuminated), and re-introduced to the same subject. In some aspects, an autologous sample (e.g., a sample from the subject to be administered) is obtained, PIT-treated and administered to the same subject.

In some embodiments, a sample is obtained from a subject, subjected to PIT (e.g., contacted with phthalocyanine-targeting molecule conjugate and illuminated), and administered to a different subject. In some aspects, a heterologous sample (e.g., a sample from a different source) is obtained, PIT-treated and administered to a subject. In some aspects, the illuminated sample (e.g., PIT-treated sample) is from both an autologous and a heterologous sample or obtained from one or more different heterologous sample.

In some embodiments, the treatment involves taking samples, e.g., containing live cells, from multiple subjects, such as a population of subjects having the same tumor type or cancer. The multiple subjects are selected, for example, to provide a representative population of mutations underlying the tumor or cancer. The samples are grown in vitro and then treated with photoimmunotherapy to induce immunogenic cell death as described herein. The collection of treated samples generates a “vaccine bank” that can be used individually or pooled together to treat other subjects with the same or similar cancers. In some embodiments, the vaccine bank includes pools of treated samples. In some embodiments, the vaccine bank includes multiple separate treated samples, such as separated by mutation type.

In some embodiments, the subject is first treated with a therapeutic treatment that may include a first therapeutic agent, prior to administering the ex vivo PIT-treated sample to the subject. In some embodiments the first therapeutic treatment is surgery. In some embodiments the first therapeutic treatment is radiation. In some embodiments, the first therapeutic agent is an anti-cancer agent. In some embodiments, the first therapeutic agent is an immune modulator, for example a checkpoint inhibitor, an anti-PD-L1 antibody, an anti-PD1 antibody, an anti-CTLA-4 antibody, an anti-CD25 antibody. In some embodiments, the first therapeutic agent is a chemotherapeutic agent. In some embodiments, the first treatment is Photoimmunotherapy using a phthalocyanine dye-targeting molecule. In some cases, the phthalocyanine dye-targeting molecule conjugate used for the first treatment is the same as the conjugate used in the ex vivo PIT treatment of the sample. In some cases, the phthalocyanine dye-targeting molecule conjugate used for the first treatment is different from the conjugate used in the ex vivo PIT treatment of the sample.

In some embodiments, the first therapeutic treatment or the first therapeutic agent is administered before the administration of the ex vivo PIT sample such as 1 day, 2 days, 3 days, 4 days, 5 days, 6 days, 7 days, 8 days, 9, days, 10 days, 11 days, 12 days, 1 week, 2 weeks, 3 weeks 4 weeks, 1 month, 2 months, 3 month, 4 months, 5 months, 6 months or more than 6 months before the administration of the ex vivo sample. In some embodiments, the first therapeutic treatment or the first therapeutic agent is administered one or more times before the administration of the ex vivo PIT sample, for example 1 times, 2 times, 3 times, 4 times, 5 times or more than 5 times.

In some embodiments, the subject is administered the ex vivo PIT-treated sample and then a second therapeutic treatment or a second therapeutic agent is administered to the subject. In some embodiments the first therapeutic treatment is surgery. In some embodiments the first therapeutic treatment is radiation. In some embodiments, the second therapeutic agent is an anti-cancer agent. In some embodiments, the second therapeutic agent is an immune modulator, for example a checkpoint inhibitor, an anti-PD-L1 antibody, an anti-PD1 antibody, an anti-CTLA-4 antibody, an anti-CD25 antibody. In some embodiments, the second therapeutic agent is a chemotherapeutic agent. In some embodiments, the second treatment is Photoimmunotherapy using a phthalocyanine dye-targeting molecule conjugate. In some cases, the photoimmunotherapy treatments uses a phthalocyanine dye-targeting molecule conjugate that is the same as the conjugate used in the ex vivo PIT treatment of the sample. In some cases, the photoimmunotherapy treatment uses a phthalocyanine dye-targeting molecule conjugate that is different from the conjugate used in the ex vivo PIT treatment of the sample.

In some embodiments, the second therapeutic treatment or agent is administered after the administration of the ex vivo PIT sample such as after 1 day, 2 days, 3 days, 4 days, 5 days, 6 days, 7 days, 8 days, 9, days, 10 days, 11 days, 12 days, 1 week, 2 weeks, 3 weeks 4 weeks, 1 month, 2 months, 3 month, 4 months, 5 months, 6 months or more than 6 months. In some embodiments, the second therapeutic treatment or agent is administered one or more times after the administration of the ex vivo sample, for example 1 times, 2 times, 3 times, 4 times, 5 times or more than 5 times.

### III. Conjugates for Use with the Methods

In some aspects, exemplary phthalocyanine dye-targeting molecule conjugate employed in the methods provided herein, include any described herein. In some aspects, other exemplary phthalocyanine dye-targeting molecule conjugate include those described in, for example, WO 2017/031363 and WO 2017/031367, which are incorporated by reference in herein.

Conjugates of a targeting molecule linked to a phthalocyanine dye for use in the compositions and methods herein include those where the targeting molecules binds to a cell surface target molecule selected from among cell membrane phospholipids, prokaryotic peptidoglycans, bacterial cell envelop proteins, viral capsid proteins, ACTHR, endothelial cell Anxa-1, aminopeptidase N, IL-6R, alpha-4-integrin, alpha-5-beta-3 integrin, alpha-5-beta-5 integrin, alpha-fetoprotein (AFP), ANPA, ANPB, APA, APN, APP, 1AR, 2AR, AT1, B1, B2, BAGE1, BAGE2, B-cell receptor BB1, BB2, BB4, calcitonin receptor, cancer antigen 125 (CA 125), CCK1, CCK2, CDS, CD10, CD11a, CD13, CD14, CD19, CD20, CD22, CD25, CD30, CD33, CD38, CD45, CD52, CD56, CD68, CD90, CD133, CD7, CD15, CD34, CD44, CD206, CD271, CEA (Carcinoembryonic Antigen), CGRP, chemokine receptors, cell-surface annexin-1, cell-surface plectin-1, Cripto-1, CRLR, CXCR2, CXCR4, DCC, DLL3, E2 glycoprotein, EGFR, EGFRvIII, EMR1, Endosialin, EP2, EP4, EpCAM, EphA2, ET receptors, FAP, Fibronectin, Fibronectin ED-B, FGFR, frizzled receptors, GAGE1, GAGE2, GAGE3, GAGE4, GAGE5, GAGE6, GLP-1 receptor, G-protein coupled receptors of the Family A (Rhodopsin-like), G-protein coupled receptors of the Family B (Secretin receptor-like), G-protein coupled receptors of the Family C (Metabotropic Glutamate Receptor-like), GD2, GP100, GP120, Glypican-3, hemagglutinin, Heparin sulfates, HER1, HER2, HER3, HER4, HMFG, HPV 16/18 and E6/E7 antigens, hTERT, IL11-R, IL-13R, ITGAM, Kalikrien-9, Lewis Y, LH receptor, LHRH-R, LPA1, MAC-1, MAGE 1, MAGE 2, MAGE 3, MAGE 4, MART1, MC1R, Mesothelin, MUC1, MUC16, Neu (cell-surface Nucleolin), Neprilysin, Neuropilin-1, Neuropilin-2, NG2, NK1, NK2, NK3, NMB-R, Notch-1, NY-ESO-1, OT-R, mutant p53, p97 melanoma antigen, NTR2, NTR3, p32 (p32/gC1q-R/HABP1), p75, PAC1, PAR1, Patched (PTCH), PDGFR, PDFG receptors, PDT, Protease-cleaved collagen IV, proteinase 3, prohibitin, protein tyrosine kinase 7, PSA, PSMA, purinergic P2X family, P2X1-5, mutant Ras, RAMP1, RAMP2, RAMPS patched, RET receptor, plexins, smoothened, sst1, sst2A, sst2B, sst3, sst4, sst5, substance P, TEMs, T-cell CD3 Receptor, TAG72, TGFBR1, TGFBR2, Tie-1, Tie-2, Trk-A, Trk-B, Trk-C, TR1, TRPA, TRPC, TRPV, TRPM, TRPML, TRPP, TRPV1-6, TRPA1, TRPC1-7, TRPM1-8, TRPP1-5, TRPML1-3, TSH receptor, VEGF receptors, VEGFR1, Flt-1, VEGFR2, FLK-1/KDR, VEGF-3, FLT-4, voltage-gated ion channels, VPAC1, VPAC2, Wilms tumor 1, Y1, Y2, Y4, and Y5.

In some embodiments, the cell surface target molecule bound by the targeting molecule of the conjugate of the composition or methods herein is selected from among HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, gangliosides (such as GD2, GD3, GM1 and GM2), VEGF receptor (VEGFR), VEGFR2, VEGF-A, integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, SK-1antigen, Bcr-abl, RET, MET, TRKB, TIE2, ALK, ROS, EML4-ALK, ROS1, BRAFV600E, SRC, c-KIT, PDGFR, mTOR, TSC1, TSC2, BTK, KIT, BRCA, CDK 4/6, JAK1, JAK2, BRAF, FLT-3, MEK1, MEK2, and SMO.

In some embodiments, the cell surface target molecule bound by the targeting molecule of the conjugate is selected from among adrenocorticotropic hormone (ACTH), angiotensin II, atrial natriuretic factor (ANF), bombesin, bradykinin, brain derived neurotrophic factor (BDNF), bone morphogenetic protein 2 (BMP-2), bone morphogenetic protein 6 (BMP-6), bone morphogenetic protein 7 (BMP-7), calcitonin, cardiotrophin 1 (BMP-2), CD22, CD40, cholecystokinin (CCK), ciliary neurotrophic factor (CNTF), CCL1-CCL28, CXCL1-CXCL17, XCL1, XCL2, CX3CL1, crypto-1 binding peptide, vascular endothelial cell growth factor (VEGF), epidermal growth factor (EGF), endothelin 1, endothelin 1/3, FAS-ligand, fibroblast growth factor 1 (FGF-1), fibroblast growth factor 2 (FGF-2), fibroblast growth factor 4 (FGF-4), fibroblast growth factor 5 (FGF-5), fibroblast growth factor 6 (FGF-6), fibroblast growth factor 1 (FGF-7), fibroblast growth factor 1 (FGF-10), Flt-3, gastrin, gastrin releasing peptide (GRP), granulocyte colony-stimulating factor (G-CSF), granulocyte macrophage stimulating factor (GM-CSF), glucagon like peptide (GLP-1), hepatocyte growth factor (HGF), interferon alpha (IFN-α), interferon beta (IFN-β), interferon gamma (IFNγ), insulin-like growth factor 1 (IGF-1), insulin-like growth factor 2 (IGF-2), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 15 (IL-15), interleukin 17 (IL-17), interleukin 19 (IL-19), luteinizing hormone (LH), luteinizing-releasing hormone (LHRH), macrophage colony-stimulating factor (M-CSF), monocyte chemotactic protein 1 (MCP-1), macrophage inflammatory protein 3a (MIP-3a), macrophage inflammatory protein 3b (MIP-3b), nerve growth factor (NGF), neuromedin B, neurotrophin 3 (NT-3), neurotrophin 4 (NT-4), neurotensin, neuropeptide Y, oxytocin, pituitary adenylate cyclase activating peptide (PACAP), platelet derived growth factor AA (PDGF-AA), platelet derived growth factor AB (PDGF-AB), platelet derived growth factor BB (PDGF-BB), platelet derived growth factor CC (PDGF-CC), platelet derived growth factor DD (PDGF-DD), netrin-1 (NTN1), netrin-2 (NTN2), netrin-4 (NTN4), netrin-G1 (NTNG1) and netrin-G2 (NTNG2), ephrin A1 (EFNA1), ephrin A2 (EFNA2), ephrin A3 (EFNA3), ephrin A4 (EFNA4), ephrin A5 (EFNA5), semaphorin 3A (SEMA3A), semaphorin 3B (SEMA3B), semaphorin 3C (SEMA3C), semaphorin 3D (SEMA3D), semaphorin 3F (SEMA3F), semaphorin 3G (SEMA3G), semaphorin 4A (SEMA4A), semaphorin 4B (SEMA4B), semaphorin 4C (SEMA4C), semaphorin 4D (SEMA4D), semaphorin 4F (SEMA4F), semaphorin 4G (SEMA4G), semaphorin 5A (SEMA5A), semaphorin 5B (SEMA5B), semaphorin 6A (SEMA6A), semaphorin 6B (SEMA6B), semaphorin 6D (SEMA6D), semaphorin 7A (SEMA7A), SLIT1, SLIT2, SLIT3, SLIT and NTRK-like family, member 1 (SLITRK1), SLIT and NTRK-like family, member 2 (SLITRK2), SLIT and NTRK-like family, member 3 (SLITRK3), SLIT and NTRK-like family, member 4 (SLITRK4), SLIT and NTRK-like family, member 5 (SLITRK5), SLIT and NTRK-like family, member 6 (SLITRK6), prostaglandin E2 (PGE2), RANTES, Somatostatin-14, Somatostatin-28, stem cell factor (SCF), stromal cell derived factor 1 (SDF-1), substance P, thyroid stimulating hormone (TSH), transforming growth factor alpha (TGF-α), transforming growth factor beta (TGF-b), tumor necrosis factor alpha (TNF-α), thrombin, vasoactive intestinal peptide (VIP), Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16, Sonic hedgehog, Desert hedgehog, and Indian hedgehog.

In some embodiments, the targeting molecule of the conjugate is an antibody, an antigen-boding fragment of an antibody or an antibody-like molecule that binds such cell surface target(s). In some embodiments, the targeting molecule of the conjugate is selected from among cetuximab, panitumumab, zalutumumab, nimotuzumab, Tositumomab (Bexxar®), Rituximab (Rituxan, MabThera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and MNRP1685A or is an antigen-binding fragment thereof. In some embodiments, the targeting molecule is an antibody or an antigen-binding antibody fragment. In some embodiments, the antibody is an antigen-binding antibody fragment that is a Fab, a single VH domain, a single chain variable fragment (scFv), a multivalent scFv, a bispecific scFv or an scFv-CH₃ dimer.

The conjugates for use with the compositions and methods herein include a phthalocyanine dye. In some embodiments, the phthalocyanine dye is a silicon phthalocyanine dye. In some embodiments, the phthalocyanine dye comprises the formula:

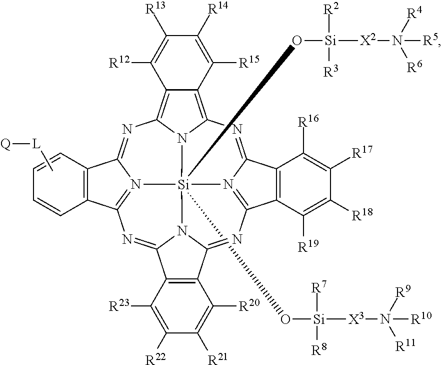

wherein:

L is a linker;

Q is a reactive group for attachment of the dye to the targeting molecule;

R², R³, R⁷, and R⁸ are each independently selected from among optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;

R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and

X² and X³ are each independently C₁-C₁₀ alkylene, optionally interrupted by a heteroatom.

In some embodiments, the phthalocyanine dye comprises the formula:

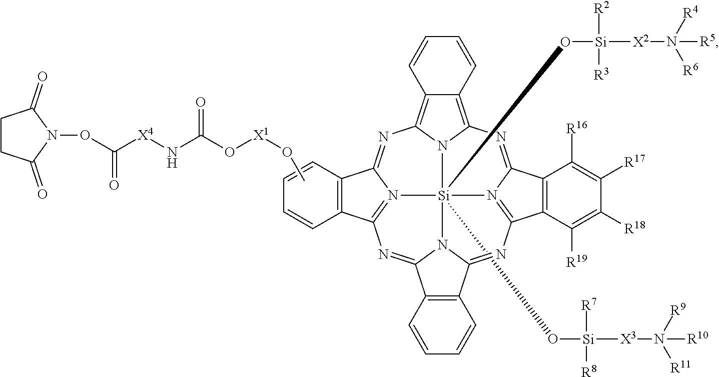

wherein:

X¹ and X⁴ are each independently a C₁-C₁₀ alkylene optionally interrupted by a heteroatom;

R², R³, R⁷, and R⁸ are each independently selected from optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and

R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments of the methods and uses provided herein, a Si-phthalocyanine dye is IRDye 700DX (IR700). In some embodiments, the phthalocyanine dye containing the reactive group is IR700 NHS ester, such as IRDye 700DX NHS ester (LiCor 929-70010, 929-70011).

In some embodiments, the dye is a compound having the following formula:

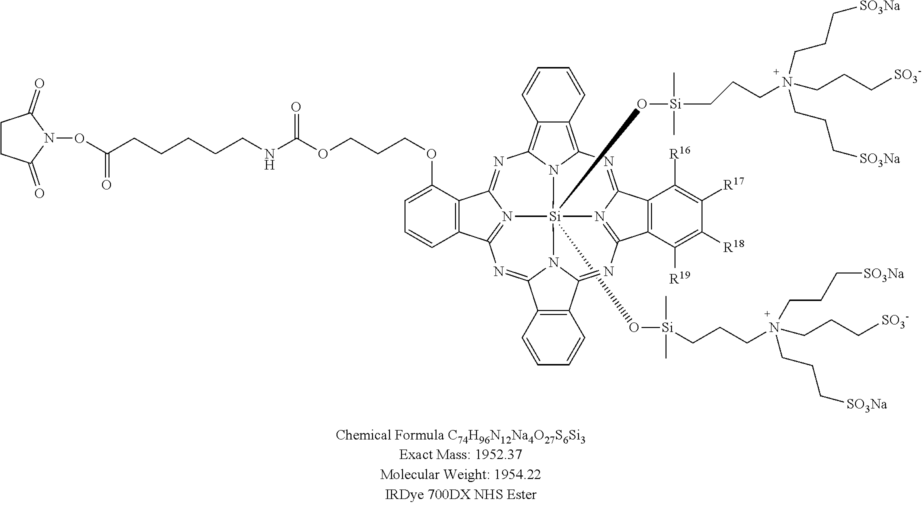

For purposes herein, the term “IR700,” “IRDye 700” or “IRDye 700DX” includes the above formula when the dye is conjugated such as to an antibody, e.g. via a reactive group.

### IV. Immune Modulators for Use with the Methods

In some embodiments of the methods herein, an immune modulatory agent is included prior to, concurrent with and/or subsequent to the ex vivo treatment. Immune modulatory agent used include an adjuvant, immune checkpoint inhibitor, cytokine or any combination thereof. In some aspects, also provided are combinations to be used in accordance with the methods and uses provided herein, that include the immune modulatory agent.

A cytokine for use in the combinations can be, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway. An adjuvant for use in the combinations can be, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

Immune checkpoint inhibitors for use in the methods herein include a PD-1 inhibitor, such as a small molecule, antibody or antigen binding fragment. Exemplary anti-PD-1 antibodies include, but are not limited to, pembrolizumab (MK-3475, Keytruda), nivolumab (OPDIVO), cemiplimab (LIBTAYO), toripalimab (JS001), HX008, SG001, GLS-010, Dostarlimab (TSR-042), Tislelizumab (BGB-A317), Cetrelimab (JNJ-63723283), pidilizumab (CT-011), genolimzumab (APL-501, GB226), BCD-100, cemiplimab (REGN2810), F520, Sintilimab (IBI308), GLS-010, CS1003, LZM009, Camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and Spartalizumab.

Immune checkpoint inhibitors for use in the methods herein include a PD-L1 inhibitor, such as a small molecule, antibody or antigen binding fragment. Exemplary anti-PD-L1 antibodies include, but are not limited to, atezolizumab (MPDL3280A, TECENTRIQ), avelumab (BAVENCIO), durvalumab (MEDI4736, IMFINZI), LDP, NM-01, STI-3031, KN035, LY3300054, M7824 (MSB0011359C), BMS-936559, MSB2311, BCD-135, BGBA333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a polypeptide linked directly or indirectly to one or more other polypeptides or chemical moieties. Such conjugates include fusion proteins, those produced by chemical conjugates and those produced by any other methods. For example, a conjugate can refer to a phthalocyanine dye, such as an IR700 molecule, linked directly or indirectly to one or more other polypeptides or chemical moieties, such as to a targeting molecule that binds to or targets to a cell surface protein.

As used herein, a composition refers to any mixture of two or more products, substances, or compounds, including cells. It may be a solution, a suspension, liquid, powder, a paste, aqueous, non-aqueous or any combination thereof.

As used herein, a “pharmaceutical composition” or “pharmaceutical formulation” refers to a preparation which is in such form as to permit the biological activity of an active ingredient contained therein to be effective, and which contains no additional components which are unacceptably toxic to a subject to which the formulation would be administered.

As used herein, a “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a derivative refers to a form of a drug that has undergone change or modification from a reference drug or agent, but still retains activity (e.g., exhibits increased or decreased activity) compared to the reference drug or agent. Typically, a derivative form of a compound means that a side chain of the compound has been modified or changed.

As used herein, an analogue or analog of a drug or agent is a drug or agent that is related to a reference drug, but whose chemical and biological activities can be different. Typically, analogues exhibit similar activities to a reference drug or agent, but the activity can be increased or decreased or otherwise improved. Typically, an analogue form of a compound or drug means that the backbone core of the structure is modified or changed compared to a reference drug.

As used herein, a kit is a packaged combination that optionally includes other elements, such as additional reagents and instructions for use of the combination or elements thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “article of manufacture” is a product that is made and, in some cases, that can be sold. In some embodiments, the term can refer to compositions contained in articles of packaging, such as in a container.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect. In particular aspects, “combination therapy” refers to a treatment in which the subject is given photoimmunotherapy (PIT), in combination with an additional therapeutic agent, such as an immune modulating agent or an anti-cancer agent. In some aspects, as used herein, “combination therapy” refers to administration of a targeting molecule-phthalocyanine dye conjugate and light treatment, in combination with an additional therapeutic agent, such as an immune modulating agent.

As used herein, “disease or disorder” refers to a pathological condition in an organism resulting from cause or condition including, but not limited to, infections, acquired conditions, genetic conditions, and characterized by identifiable symptoms.

As used herein, “treating” a subject with a disease or condition means that the subject's symptoms are partially or totally alleviated or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### VI. Exemplary Embodiments

Among the Provided Embodiments are:

1. A method of treating a tumor or a lesion comprising:

administering to a first subject a composition comprising an illuminated sample, wherein the illuminated sample comprises tumor cells that have been treated ex vivo with photoimmunotherapy, wherein the photoimmunotherapy comprises:

i) contacting a sample of tumor cells ex vivo with a conjugate comprising a phthalocyanine dye linked to a targeting molecule; and

ii) after contacting with the conjugate, illuminating the sample at a wavelength of at or about 600 nm to at or about 850 nm to obtain the illuminated sample.

2. A method of treating a tumor or a lesion comprising:

administering to a first subject a composition comprising an illuminated sample, wherein the illuminated sample comprises tumor cells that have been treated ex vivo with photoimmunotherapy, wherein the photoimmunotherapy comprises:

i) contacting a sample of tumor cells ex vivo with a conjugate comprising a silicon phthalocyanine dye linked to a targeting molecule; and

ii) after contacting with the conjugate, illuminating the sample at a wavelength of at or about 600 nm to at or about 850 nm to obtain the illuminated sample.

3. The method of embodiment 1 or 2, wherein the tumor cells or a portion thereof within the illuminated sample exhibit one or more markers of immunogenic cell death (ICD).

4. A method of treating a tumor or a lesion comprising:

administering to a first subject a composition comprising an illuminated sample, wherein the illuminated sample comprises tumor cells that have been treated ex vivo with photoimmunotherapy, wherein the photoimmunotherapy comprises:

i) contacting a sample of tumor cells ex vivo with a conjugate comprising a silicon phthalocyanine dye linked to a targeting molecule; and

ii) after contacting with the conjugate, illuminating the sample at a wavelength of at or about 600 nm to at or about 850 nm to obtain the illuminated sample,

wherein the tumor cells or a portion thereof within the illuminated sample exhibit one or more markers of immunogenic cell death (ICD).

5. A method of treating a tumor or a lesion comprising:

contacting a sample of tumor cells ex vivo with a conjugate comprising a phthalocyanine dye linked to a targeting molecule;

after contacting with the conjugate, illuminating the sample at a wavelength of at or about 600 nm to at or about 850 nm to obtain an illuminated sample; and

administering a composition comprising the illuminated sample to a first subject.

6. A method of treating a tumor or a lesion comprising:

contacting a sample of tumor cells ex vivo with a conjugate comprising a silicon phthalocyanine dye linked to a targeting molecule;

after contacting with the conjugate, illuminating the sample at a wavelength of at or about 600 nm to at or about 850 nm to obtain an illuminated sample; and

administering a composition comprising the illuminated sample to a first subject.

7. The method of embodiments 5 or 6, wherein the tumor cells or a portion thereof within the illuminated sample exhibit one or more markers of immunogenic cell death (ICD).

8. A method of treating a tumor or a lesion comprising:

contacting a sample of tumor cells ex vivo with a conjugate comprising a silicon phthalocyanine dye linked to a targeting molecule;

after contacting with the conjugate, illuminating the sample at a wavelength of at or about 600 nm to at or about 850 nm to obtain an illuminated sample; and

administering a composition comprising the illuminated sample to a first subject,

wherein the tumor cells or a portion thereof within the illuminated sample exhibit one or more markers of immunogenic cell death (ICD).

9. The method of any of embodiments 3, 4, 7, and 8, wherein the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

10. The method of any of embodiments 1-9, wherein the composition exhibits less than 100% cell death prior to administration.

11. The method of any of embodiments 1-10, wherein the composition exhibits between about 30% and about 70% cell death prior to administration.

12. The method of any of embodiments 1-11 wherein the sample comprises tumor cells derived or obtained from the first subject.

13. The method of any of embodiments 1-11, wherein the sample comprises tumor cells derived or obtained from a second subject.

14. The method of any of embodiments 1-13, wherein the sample comprises tumor cells that have been grown or cultivated in vitro prior to contacting with the conjugate.

15. The method of embodiment 14, wherein the sample comprises tumor cells that have been grown or cultivated into an organoid prior to contacting with the conjugate.

16. The method of any of embodiments 1-15, wherein the composition is treated to prevent cell growth or cell expansion prior to administration to the first subject.

17. The method of embodiment 16, wherein the composition is treated by irradiation to prevent cell growth or cell expansion.

18. The method of embodiment 17, wherein the irradiation comprises gamma irradiation.

19. The method of embodiment 1-18, wherein the first subject has been diagnosed as having, or is suspected of having, a type of cancer.

20. The method of embodiment 19, wherein the sample comprises tumor cells that are derived from the same or similar type of cancer.

21. The method of embodiment 19 or 20, wherein the type of cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, cancer of the blood, leukemia, lymphoma, and multiple myeloma, and any combination thereof.

22 The method of any of embodiments 1-21, wherein administering the composition results in a stimulation of an anti-cancer immune response in the first subject.

23. The method of any of embodiments 1-22, wherein administering the composition results in a reduction of growth, a reduction is size, a reduction in volume, or elimination of a tumor, a lesion or a metastasis in the first subject.

24. The method of any of embodiments 1-23, wherein the composition is administered by injection or by infusion to the first subject.

25. The method of any of embodiments 1-23, wherein the composition is administered by implantation into the first subject.

26. The method of any of embodiments 1-25, wherein the targeting molecule comprises an antibody or an antigen binding fragment thereof.

27. The method of any of embodiments 1-26, wherein the targeting molecule binds to a cell surface molecule.

28. The method of embodiment 27, wherein the cell surface molecule is present on a first tumor cell or a first cell in the tumor microenvironment, optionally wherein the sample comprises the first tumor cell or the first cell in the tumor microenvironment.

29. The method of embodiment 27 or 28, wherein the cell surface molecule is selected from the group consisting of HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, a ganglioside, VEGF receptor (VEGFR), VEGFR2, VEGF-A, integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, SK-1antigen, Bcr-abl, RET, MET, TRKB, TIE2, ALK, ROS, EML4-ALK, ROS1, BRAFV600E, SRC, c-KIT, PDGFR, mTOR, TSC1, TSC2, BTK, KIT, BRCA, CDK 4/6, JAK1, JAK2, BRAF, FLT-3, MEK1, MEK2, and SMO.

30. The method of any of embodiments 1-29, wherein the targeting molecule is selected from the group consisting of cetuximab, panitumumab, zalutumumab, nimotuzumab, Tositumomab (Bexxar®), Rituximab (Rituxan, MabThera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002, and MNRP1685A, and any antigen-binding fragment thereof.

31. The method of any of embodiments 2-4, and 6-30, wherein the silicon phthalocyanine dye is IR700.

32. The method of any of embodiments 1-31, further comprising administering a second treatment to the first subject, wherein the second treatment comprises:

administering to the first subject a second conjugate comprising a second phthalocyanine dye linked to a second targeting molecule, and

after administering the second conjugate, illuminating a tumor or a lesion in the first subject at a wavelength of at or about 600 nm to at or about 850 nm and at a dose of from at or about 25 J/cm² to at or about 400 J/cm² or from at or about 2 J/cm fiber length to at or about 500 J/cm fiber length.

33. The method of embodiment 32, wherein the second treatment is administered subsequent to administering the composition to the first subject.

34. The method of embodiment 32, wherein the second treatment is administered prior to administering the composition to the first subject.

35. The method of embodiment 32, wherein the second treatment is administered prior to and subsequent to administering the composition to the first subject.

36. The method of any of embodiments 1-35, wherein the composition is administered in combination with an immune modulatory agent.

37. The method of embodiment 36, wherein the immune modulatory agent administered prior to, concurrent with and/or subsequent to the composition.

38. The method of embodiment 36 or 37, wherein the immune modulatory agent comprises an adjuvant, an immune checkpoint inhibitor, a cytokine or any combination thereof.

39. The method of any of embodiments 1-38, wherein the tumor cells are obtained from multiple tumor sources.

40. The method of any of embodiments 1-39, wherein the sample comprises tumor cells from a tumor or a lesion associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.

41. A pharmaceutical composition, comprising photoimmunotherapy-treated tumor cells formulated with at least one pharmaceutically acceptable excipient.

42. The pharmaceutical composition of embodiment 41, wherein the tumor cells or a portion thereof in the pharmaceutical composition exhibit one or more markers of immunogenic cell death (ICD).

43. The pharmaceutical composition of embodiment 41 or 42, wherein the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

44. The pharmaceutical composition of any of embodiments 41-43, wherein the tumor cells within the pharmaceutical composition exhibit less than 100% cell death.

45. The pharmaceutical composition of any of embodiments 41-44, wherein the tumor cells within the pharmaceutical composition exhibit between about 30% and about 70% cell death.

46. The pharmaceutical composition of any of embodiments 41-45, comprising tumor cells derived from a single subject.

47. The pharmaceutical composition of any of embodiments 41-45, comprising tumor cells derived from more than one subject.

48. The pharmaceutical composition of any of embodiments 41-47, comprising tumor cells derived from a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.

49. The pharmaceutical composition of any of embodiments 41-48, for use in treating a tumor or a lesion.

50. Use of the pharmaceutical composition of any of embodiments 41-48, in treating a tumor or a lesion.

51. Use of the pharmaceutical composition of any of embodiments 41-48, in the manufacture of a medicament for treating a tumor or a lesion.

52. A method of treating a tumor or a lesion comprising:

obtaining a sample of cells;

contacting the sample with a conjugate comprising a phthalocyanine dye linked to a targeting molecule;

after contacting with the conjugate, illuminating the sample at a wavelength of at or about 600 nm to at or about 850 nm to obtain an illuminated sample;

administering the illuminated sample to a first subject.

53. The method of any of embodiments 1-40, and 52, wherein the sample of cells is obtained from a biopsy of the first subject.

54. The method of any of embodiments 1-40, and 52, wherein the sample of cells is obtained from a second subject.

55. The method of any of embodiments 1-40, and 52-54, further comprising growing or cultivating the sample in vitro prior to contacting with the conjugate.

56. The method of any of embodiments 1-40, and 55, wherein the sample is grown or cultivated into an organoid prior to contacting with the conjugate.

57. The method of any of embodiments 1-40, and 52-56, wherein the illuminated sample is administered by injection or by infusion to the first subject.

58. The method of any of embodiments 1-40, and 52-56, wherein the illuminated sample is administered by implantation into the first subject.

59. The method of any of embodiments 1-40, and 52-58, wherein the targeting molecule comprises an antibody or an antigen binding fragment thereof.

60. The method of any of embodiments 1-40, and 52-59, wherein the targeting molecule binds to a cell surface molecule.

61. The method of any of embodiments 1-40, and 60, wherein the cell surface molecule is present on a tumor cell or a cell in the tumor microenvironment, optionally wherein the sample of cells comprises the tumor cell or the cell in the tumor microenvironment.

62. The method of any of embodiments 1, 5, and 52-61, wherein the phthalocyanine dye is a silicon phthalocyanine dye.

63. The method of any of embodiments 1-40, and 62, wherein the silicon phthalocyanine dye is IR700.

64. The method of any of embodiments 1-40, and 52-63, wherein the targeting molecule is selected from the group consisting of cetuximab, panitumumab, zalutumumab, nimotuzumab, Tositumomab (Bexxar®)), Rituximab (Rituxan, MabThera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and MNRP1685A and any antigen-binding fragment thereof.

65. The method of any of embodiments 52-64, further comprising administering a second treatment to the first subject, wherein the second treatment comprises:

administering to the first subject a second conjugate comprising a second phthalocyanine dye linked to a second targeting molecule, and

after administering the second conjugate, illuminating a tumor or a lesion in the first subject at a wavelength of at or about 600 nm to at or about 850 nm and at a dose of from at or about 25 J/cm² to at or about 400 J/cm² or from at or about 2 J/cm fiber length to at or about 500 J/cm fiber length.

66. The method of any of embodiments 1-40, and 65, wherein the second treatment is administered subsequent to the administration of the illuminated sample to the first subject.

67. The method of any of embodiments 1-40, and 65, wherein the second treatment is administered prior to the administration of the illuminated sample to the first subject.

68. The method of any of embodiments 1-40, and 65, wherein the second treatment is administered prior to and subsequent to the administration of the illuminated sample to the first subject.

69. The method of any of embodiments 1-40, and 52-68, wherein the illuminated sample is administered in combination with an immune modulatory agent.

70. The method of any of embodiments 1-40, and 69, wherein the immune modulatory agent is administered prior to, concurrent with and/or subsequent to the illuminated sample.

71. The method of any of embodiments 1-40, and 69 or any of embodiments 1-40, and 70, wherein the immune modulatory agent comprises an adjuvant, an immune checkpoint inhibitor, a cytokine or any combination thereof.

72. The method of any of embodiments 1-40, and 52-71, wherein the sample of cells is obtained from a tumor.

73. The method of any of embodiments 1-40, and 52-72, wherein the sample of cells comprises tumor cells.

74. The method of any of embodiments 1-40, and 52-73, wherein the tumor cells are obtained from multiple tumor sources.

75. The method of any of embodiments 1-40, and 52-74, wherein the first subject has, has been diagnosed as having, or is suspected of having a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

76. The method of any of embodiments 1-40, and 52-75, wherein the sample of cells comprises cells from a tumor or a lesion associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

77. The method of any of embodiments 1-40, and 52-76, wherein the illuminated sample comprises at least 30%-70% cell death prior to administration.

78. The method of any of embodiments 1-40, and 52-77, wherein the sample is treated to prevent cell growth or cell expansion prior to administration to the first subject.

79. The method of any of embodiments 1-40, and 78, wherein the sample is treated to prevent cell growth or cell expansion prior to administration to the first subject.

80. The method of any of embodiments 1-40, and 79, wherein the irradiation is gamma irradiation.

### VII. Examples

The following examples are included for illustrative purposes only and are not intended to limit the scope of the invention.

### Example 1: Release and Expression of Immunogenic Cell Death (ICD) Indicators

Release and expression of several proteins indicative of immunogenic cell death (ICD) were evaluated after photoimmunotherapy (PIT) treatment by incubation with exemplary targeting molecule-phthalocyanine dye conjugates and light illumination.

A. Annexin A1 (ANXA1) Release

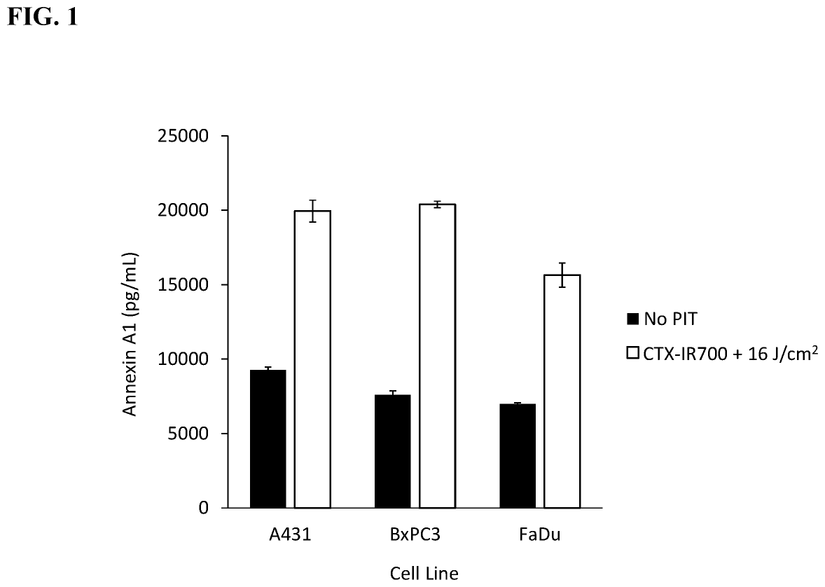

EGFR-expressing A-431 epidermoid carcinoma (ATCC® CRL-1555), BxPC3 pancreatic cancer (ATCC® CRL-1687), and FaDu (ATCC® HTB43) squamous cell carcinoma cells were incubated with an exemplary antibody-IR700 conjugate, anti-EGFR (cetuximab)-IRDye 700DX (CTX-IR700), and subjected to light illumination (PIT). A-431, BxPC3 and FaDu cells were illuminated at 16 J/cm², with a 690 nm laser. Levels of ANXA1 released in the supernatant were measured using a MesoScaleDiscovery ANXA1 assay. As shown in FIG. 1, 24 h post treatment, PIT led to robust Annexin A1 (ANXA1) release from the cells, compared to the control groups that were not illuminated. The results showed that the release of ANXA1 increased upon PIT-mediated target cell death, consistent with PIT-treated cells exhibiting characteristics of ICD and having the potential to activate an immune response.

B. Adenosine Triphosphate (ATP) Release

In some aspects, release of ATP from cancer cells is consistent with immunogenic cell death (ICD), and can be an attractant for macrophages and dendritic cells.

4T1 murine mammary gland cells, engineered to express epithelial cell adhesion molecule (4T1-EpCam), A-431 epidermoid carcinoma, BxPC3 pancreatic adenocarcinoma, and CT26 murine colon carcinoma cells engineered to express Ephrin type-A receptor 2 (CT26-EphA2), and LL/2 mouse lung carcinoma cells expressing EphA2 were incubated with three exemplary antibody-IR700 conjugates (anti-EpCam-IR700 incubated with 4T1-EpCam cells, anti-EGFR-IR700 with A-431, BxPC3 and FaDu cells, and anti-EphA2-IR700 with CT26-EphA2 and LL/2-EphA2) and subjected to illumination. ATP, released into the medium, was measured by a bioluminescent plate assay.

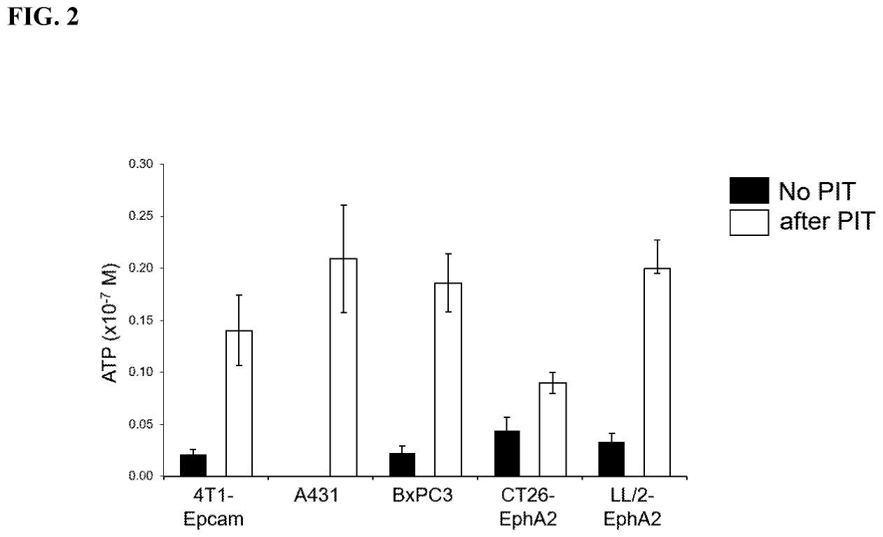

As shown in FIG. 2, incubation with the antibody-IR700 conjugates and illumination led to a rapid and extensive ATP release from the cells, compared to the control groups that were not illuminated. The results showed that the release of ATP was substantially increased upon PIT-mediated target cell death in 5 different cancer cell lines using 3 different antibody-IR700 conjugates, consistent with PIT-treated cells exhibiting characteristic of ICD and having the potential to activate an immune response.

In a further study, 4T1-EpCam, A-431, BxPC3, CT26-EphA2, FaDu human squamous cell carcinoma, and LL/2-EphA2 cells were incubated with anti-EpCam-IR700, anti-EGFR-IR700, or anti-EphA2-IR700 (same cell-antibody conjugate combinations as above, and FaDu incubated with anti-EGFR-IR700) and subjected to illumination as described above. ATP released by the cells was measured by bioluminescent assay and compared to ATP released from untreated cells at 0, 1, 3, 6, and 26 hours post illumination. At the same time points, cell death of treated cells was measured by CellToxGreen assay (Promega).

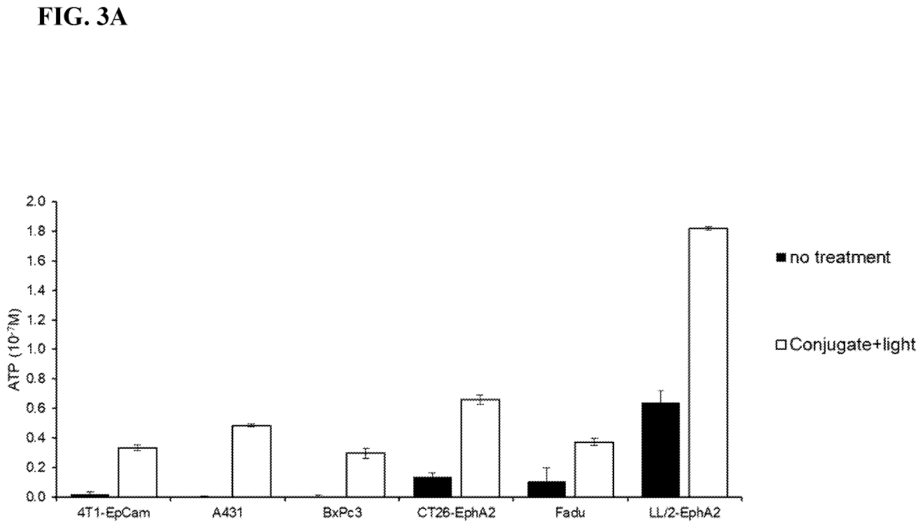

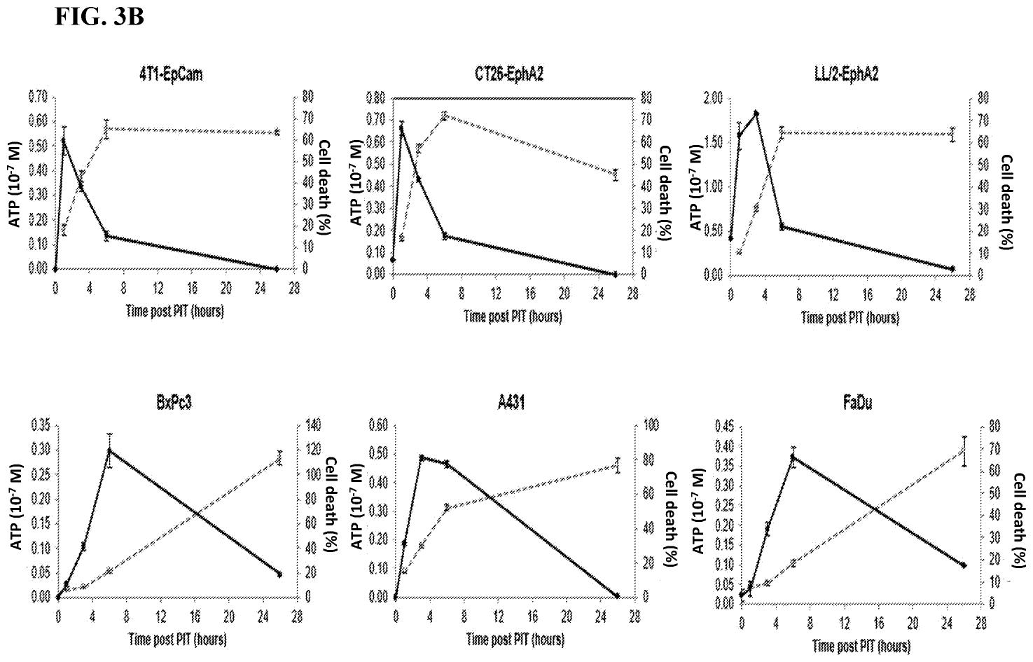

ATP release from treated and untreated cells at the 26 hour time point, shown in FIG. 3A, confirmed that the tested cancer cells release ATP in response to PIT treatment. The time course of the ATP release (solid line; left axis) and percent cell death (dashed line; right axis) of PIT-treated cells is shown in FIG. 3B. These results show that ATP is released as the cells are dying following PIT treatment, but the maximal ATP release occurs hours before the maximal cell death, implying an active mechanism of ATP secretion in response to PIT.

C. Interferon α and Interferon β

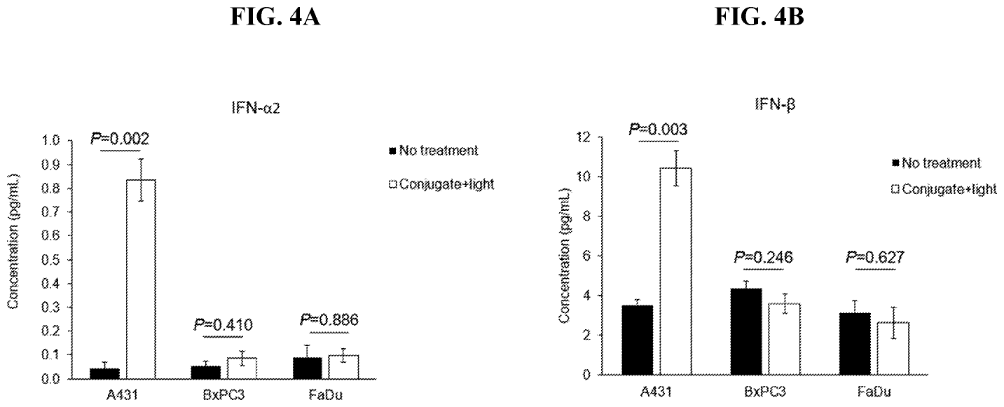

In some aspects, release of interferon-α proteins (IFNα) and/or interferon-β (IFNβ) from cancer cells is consistent with immunogenic cell death (ICD), and can enhance innate and adaptive cell functionality. A-431, BxPC3, and FaDu cells were incubated with anti-EGFR-IR700 and subjected to illumination, to effect PIT treatment, as described above. Untreated cells served as a control. 1 hour after treatment, IFN-α2 and IFN-β were measured in the medium of treated and untreated cells using a MesoScaleDiscovery IFN-α2 and IFN-β assay. Following PIT treatment, A-431 epidermoid carcinoma cells released significantly more IFN-α2 and IFN-β than untreated cells (FIGS. 4A and 4B, respectively). A similar increase was not observed for the other cell lines tested.

D. High-Mobility Group Protein B1 (HMGB1) Release

EGFR-expressing A-431 (ATCC® CRL-1555™) epidermoid carcinoma and FaDu (ATCC® HTB-43™) squamous cell carcinoma cells were incubated with anti-EGFR-IRDye 700DX. The tumor cells were illuminated at 32 J/cm² for PIT-mediated killing of target cells, and the culture supernatants were assessed for secretion of HMGB1 using an enzyme-linked immunosorbent assay (ELISA). Cells not treated with light served as controls.

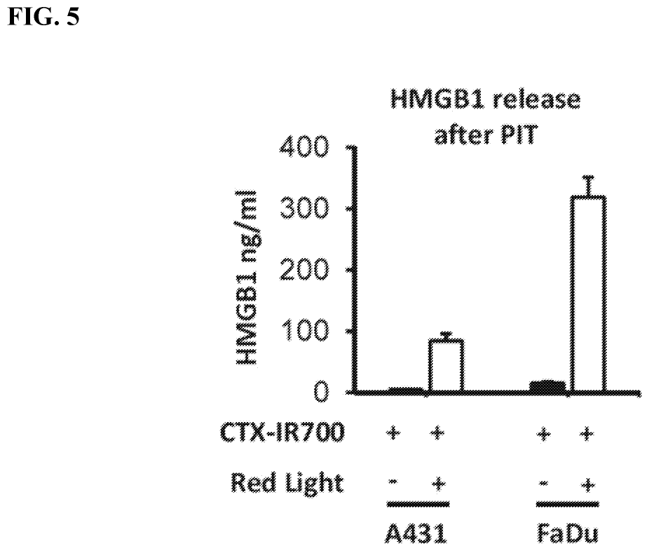

As shown in FIG. 5, PIT treatment after incubation of EGFR-expressing A-431 and FaDu cells with CTX-IR700 resulted in a large increase of release of the nuclear ICD marker HMGB1 into the supernatant of the culture, compared to the control groups that did not receive light treatment.

E. Surface Expression of Heat Shock Proteins and Calreticulin (CRT)

Cell surface expression of HSP70, HSP90, and calreticulin (CRT) was evaluated after incubation with an exemplary antibody-IR700 conjugate and light treatment (PIT) as additional indicators of ICD.

EGFR-expressing A-431 (ATCC® CRL-1555™) epidermoid carcinoma and FaDu (ATCC® HTB-43™) squamous cell carcinoma cells were incubated with anti-EGFR-IRDye 700DX. A-431 cells were illuminated at 6 J/cm² and FaDu cells were illuminated at 12 J/cm², with a 690 nm laser. Cell surface expression of HSP70, HSP90, and calreticulin (CRT), were measured by flow cytometry after staining with antibodies specific for each protein. Cells not treated with light served as controls.

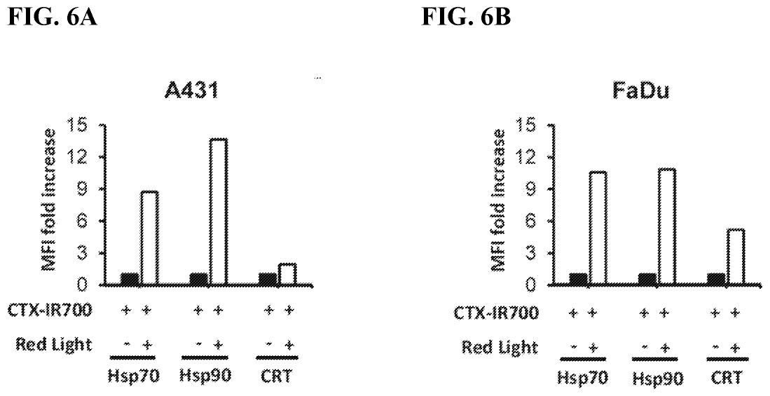

As shown in FIGS. 6A and 6B, PIT treatment after incubation of EGFR-expressing A-431 and FaDu cells with CTX-IR700 exhibited increased expression of exemplary ICD markers HSP70, HSP90 and calreticulin (CRT) compared to control groups that did not receive light treatment.

The results above showed that expression or release of various ICD markers were increased upon PIT-mediated target cell death after incubation with the exemplary CTX-IR700 conjugates and light treatment, consistent with PIT-treated cells exhibiting markers characteristic of ICD and having the potential to activate immune cells.

### Example 2: Activation of Dendritic Cells Upon Killing of Target Cells after PIT

Activation of dendritic cells (DCs) was assessed after exposure to PIT-treated tumor cells. As described in the Example 1 above, PIT-treated cells undergo immunogenic cell death, exhibiting elevated release of ATP and HMGB1, which can enhance stimulation or activation of immune cells such as DCs. As DCs become activated, surface expression of DC maturation/activation markers, such as cluster of differentiation 86 (CD86) and major histocompatibility complex II (MHCII), can be elevated, and can result in production of pro-inflammatory cytokines.

Human DCs were exposed to target cancer cells incubated with the exemplary anti-EGFR-IRDye 700DX conjugate, with or without light treatment, generally as described in Example 1 above. Following exposure to the target cancer cells, the DCs were assessed by flow cytometry for expression of activation markers CD86 and MHCII, and production of pro-inflammatory cytokines such as tumor necrosis factor (TNF), IFN-γ-Inducible Protein 10 (IP-10), MIP-1α (Macrophage Inflammatory Protein-1 alpha), MIP-1β (Macrophage Inflammatory Protein-1 beta), interleukin-1 beta (IL-1β) and interleukin-8 (IL-8) was assessed using a multiplexed immunoassay.

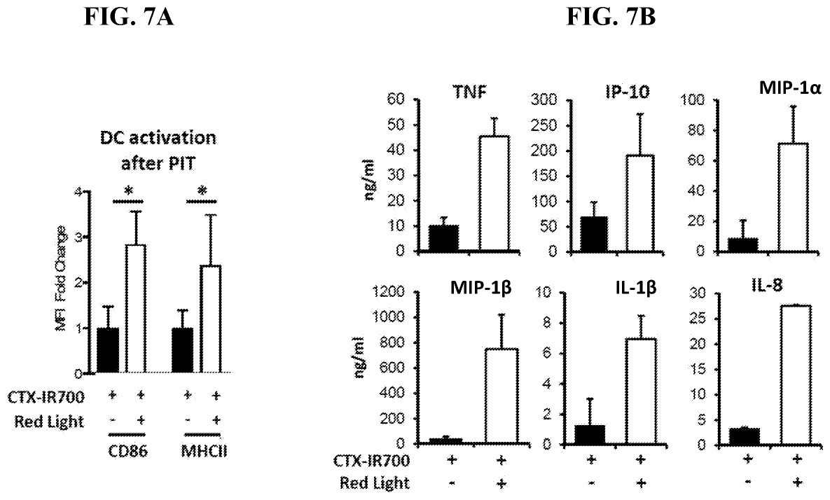

As shown in FIG. 7A, human DCs exposed to PIT-treated tumor cells exhibited higher expression of dendritic cell activation markers CD86 and MHCII, compared to DCs exposed to supernatant from control cells without light treatment. As shown in FIG. 7B, human DCs produced higher amounts of several pro-inflammatory cytokines, including TNF, IP-10, MIP-1α, MIP-1β, IL-1β and IL-8, after exposure to PIT-treated tumor cells. The results showed that immune cells, such as DCs, can be activated and secrete pro-inflammatory cytokines upon exposure to cancer cells killed by PIT using antibody-IR700 conjugates.

### Example 3: Photoimmunotherapy and Immune Activation

To determine the contribution of the immune response to photoimmunotherapy anticancer activity, photoimmunotherapy (PIT) treatment was applied to immunocompetent mice in conjunction with blockade of CD40-CD40L axis at various timepoints or with antibody-mediated CD8 T-cell depletion.

A. Pre-Treatment Blockade of CD40-CD40L

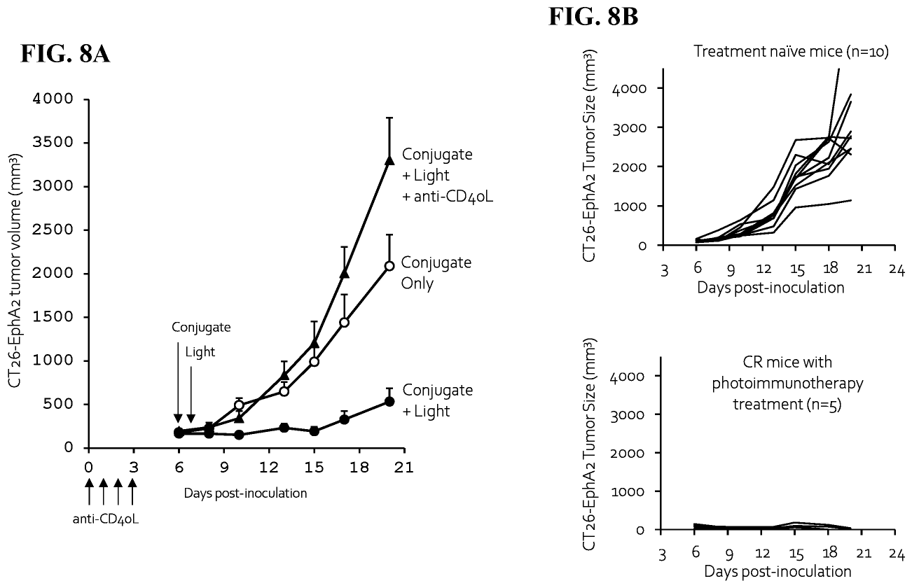

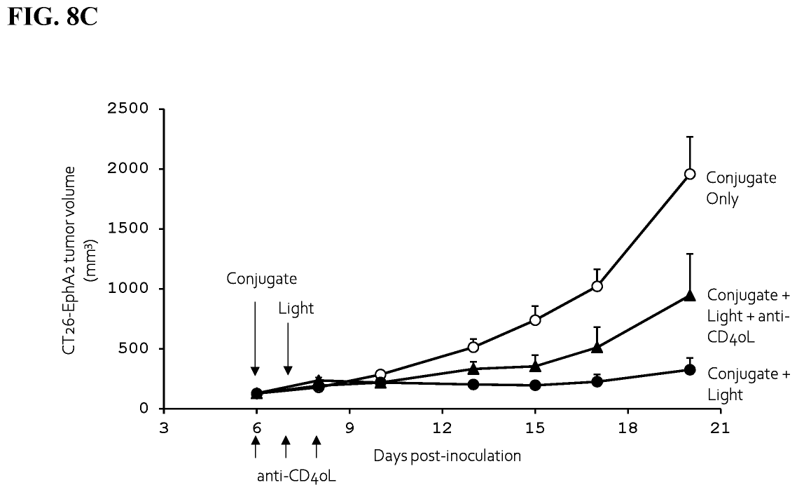

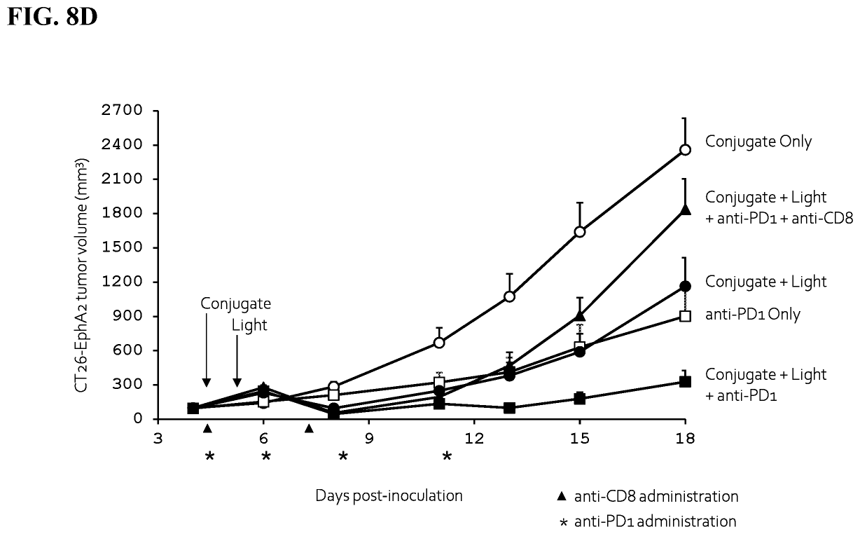

A syngeneic mouse model was established by subcutaneous inoculation of 1×10⁶ CT26-EphA2 murine colon carcinoma cells engineered to express Ephrin type-A receptor 2 (CT26-EphA2) in the right hind flank of female BALB/c mice (30 mice total). To determine the effect of pre-existing immunity on photoimmunotherapy treatment, 10 out of the 30 mice were dosed with anti-CD40L antibody starting on the day of inoculation (day 0) and on day 1, 2, and 3 after inoculation. On day 6, all mice were administered anti-EphA2-IR700 conjugate. Twenty-four±2 hours after conjugate dosing (day 7), the tumors of the 10 mice having received the anti-CD40L antibody and 10 out of the 20 mice having received only the conjugate were illuminated at 690 nm at a dosage of 100 J/cm². Ten mice receiving only the conjugate were not illuminated. Average tumor volume was measured until day 20 (FIG. 8A).

Mice administered only with anti-EphA2 conjugate exhibited tumor growth over the course of the study (FIG. 8A; open circles). Mice receiving PIT (conjugate+light) exhibited markedly reduced tumor growth (FIG. 8A; closed circles) compared to conjugate treatment alone (open circles), with 5/10 mice achieving a complete response (CR). Blocking CD40L using anti-CD40L antibody completely abrogated the anti-cancer activity of PIT (FIG. 8A; closed triangles).

B. Tumor Re-Challenge

On day 42 the mice that achieved complete response (CR) following PIT treatment (n=5) in Part A above were re-challenged with CT26-EphA2 tumor cells, and treatment naïve mice were inoculated with CT26-EphA2 cells, as described above to serve as controls (n=10). Tumor growth was monitored for 20 days. The tumor growth of individual mice is plotted in FIG. 8B.

Treatment naïve mice developed tumors that increased in size over the course of the study (FIG. 8B; top panel), whereas all of the previously CR PIT-treated mice rejected the tumor re-challenge (FIG. 8B; bottom panel). These results indicate that PIT treatment enhanced the adaptive immune response and permitted resistance to subsequent tumor challenge.

C. Post-Treatment Blockade of CD40-CD40L

Mice were inoculated with CT26-EphA2 cells by subcutaneous injection as described in part A above (n=30). To examine the anticancer activity involving new T-cell priming, 10 mice were dosed with anti-CD40L on day 6, 7, and 8 after inoculation. Anti-EphA2-IR700 conjugate was administered to all mice 6 days after inoculation. Twenty-four±2 hours after conjugate dosing, 100 J/cm² of 690 nm light was applied to the tumor of the photoimmunotherapy treated groups. Tumor growth was measured for 20 days.

As shown in FIG. 8C, mice administered conjugate alone exhibited continuous tumor growth over the course of the study, while mice treated by PIT (conjugate+light) exhibited limited tumor growth (FIG. 8C; open circles vs. closed circles, respectively). Administration of anti-CD40L in addition to PIT resulted in diminished anti-cancer activity (FIG. 8C; closed triangles), indicating that the immune response induced by PIT includes priming of new T cells, and demonstrating that the immune response triggered by PIT plays an important role in the efficacy of photoimmunotherapy.

D. CD8 T-Cell Depletion

Mice were inoculated with CT26-EphA2 cells by subcutaneous injection as described in part A above (n=50). On day 4 and 7 after inoculation, 10 mice were dosed with anti-CD8 to eliminate CD8⁺ T cells. In mice receiving the conjugate, anti-EphA2-IR700 was administered 4 days post-inoculation. Twenty-four±2 hours after conjugate dosing, 100 J/cm² of 690 nm light was applied to the tumor of the PIT-treated groups. Mice receiving anti-PD1 were administered the antibody on days 4, 6, 8 and 11 after inoculation. Tumor growth was monitored for 18 days.

Mice treated with conjugate alone exhibited continuous tumor growth over the course of the study (FIG. 8D; open circles). Mice receiving PIT only (conjugate+light) or anti-PD1 only exhibited reduced tumor growth (FIG. 8D; closed circles and open squares, respectively). The effects of PIT were enhanced by combining PIT with anti-PD1 therapy (FIG. 8D; closed squares) Eliminating CD8⁺ T cells by administering anti-CD8 antibody substantially reduced the anti-cancer activity of PIT+anti-PD1 therapy (FIG. 8D; closed triangles versus closed squares, respectively). These results indicate that the efficacy of PIT+anti-PD1 therapy is dependent on CD8 T-cell activity. Together with results from Parts A, B, and C, these results confirm the importance of the adaptive immune system for the anticancer effects of PIT.

### Example 4: Cancer Vaccination with Cancer Cells Subject to Ex Vivo PIT Using Targeting Molecule-Phthalocyanine Dye Conjugates

This example describes an exemplary cancer vaccination by administering cancer cells that have been subject to ex vivo photoimmunotherapy (PIT) by incubation with exemplary targeting molecule-phthalocyanine dye conjugates and light illumination.

CT26 murine colon carcinoma cells, engineered to express Ephrin type-A receptor 2 (CT26-EphA2), were incubated with an exemplary antibody-IR700 conjugate, that specifically binds to EphA2 for 1 hour in 1-STACK Chamber (Corning). The cells were then trypsinized, resuspended in PBS in a conical tube, illuminated at 50 J/cm using a 2 cm fiber with a radial diffuser inserted in the center of the tube (PIT group), and re-plated. As a control, plated CT26-EphA2 cells were incubated with 150 μM cisplatin (cisplatin group). The cytotoxicity was monitored for both groups, and when the cells achieved 50% cell death, the dying PIT-treated and cisplatin-treated CT26-EphA2 cell samples were injected subcutaneously into mice (1.5×10⁶ cells in 200 μL/mouse). One week later, mice were then challenged by flank-implantation of live, untreated CT26-EphA2 tumor cells, and the volume of the introduced tumors was monitored over time.

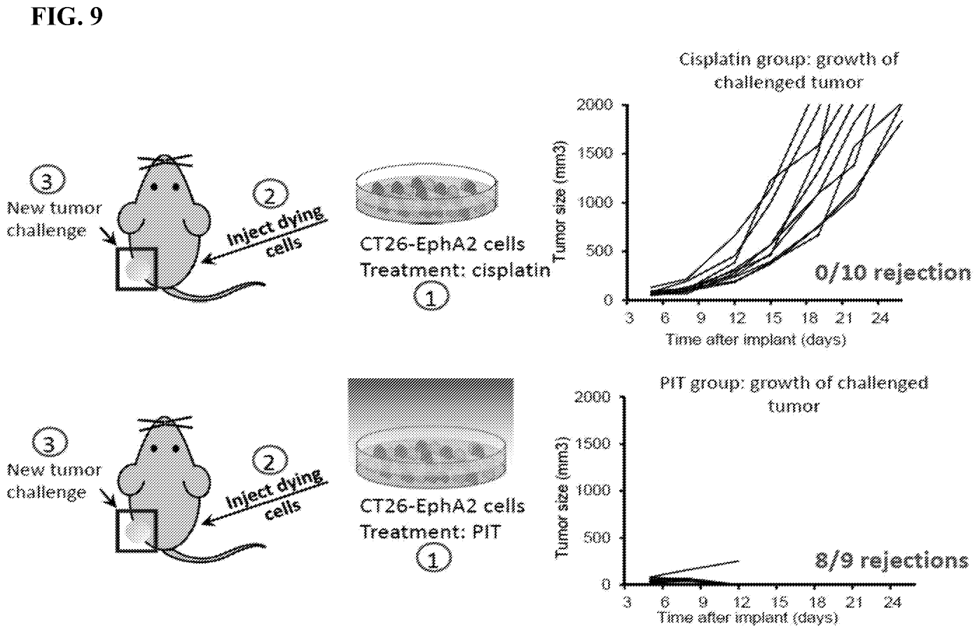

As shown in FIG. 9, eight (8) out of nine (9) mice that had been administered PIT-treated samples (PIT group) rejected the challenge tumor, and the tumor disappeared. In contrast, in all 10 mice that had been administered samples treated with cisplatin (cisplatin group), the volume of the introduced tumor increased substantially over time. These results were confirmed by a further study (data not shown). The results showed that the administered PIT-treated samples vaccinated the subjects against cancer. The results were consistent with the utility of an exemplary method employing administering a tumor sample that has been subjected to PIT using an exemplary targeting molecule-phthalocyanine dye conjugate and light illumination, for stimulating, enhancing or augmenting an anticancer immune response in the subject and leading to cancer vaccination. The rejection of tumor cells following vaccination with PIT-treated cells, but not cisplatin treated cells, demonstrates that PIT-induced ICD, not merely cell toxicity, elicits an immune response in the treated subject which resulted in tumor resistance.

### Example 5: Cell Viability Status and PIT-Mediated Tumor Immunity

Completely expired PIT-treated CT26-EphA2 cells (100% cell death; dead cells) were examined for their ability to confer PIT-mediated immunity to CT26-EphA2 tumor growth compared to cells still in the process of undergoing PIT-mediated cell death (50% cell death; dying cells) or saline only treatment. To this end, CT26-EphA2 cells were subjected to ex vivo PIT therapy and injected subcutaneously into mice (1.5×10⁶ in 200 μL/mouse), approximately 4 hours (dying cells: 50% cell death) or approximately 24 hours (dead cells: 100% cell death) after irradiation (n=10 for each condition). Mice were also injected with saline only as a control (n=10). One week later, mice were challenged by flank-implantation of live, untreated CT26-EphA2 tumor cells. The volumes of the introduced tumors and tumor-free survival were measured every 2-5 days for 23 days.

Tumor volumes of individual mice are plotted in FIGS. 10A-10C as described below, the average tumor volumes are plotted in FIG. 10D, and the tumor-free survival is depicted in FIG. 10E. 9 of the 10 mice that were administered PIT-treated CT26-EphA2 cells and challenged with the same CT26-EphA2 cells, rejected the tumor, and the tumor disappeared (FIG. 10A). In contrast, 8 of the 10 mice administered cells treated with dead PIT-treated cells, exhibited tumor growth, with the volume of the tumor increasing over the course of the study (FIG. 10B). Similar results were observed for the saline-treated mice (FIG. 10C). The average tumor growth and tumor-free survival from mice administered dead PIT-treated cells was similar to saline-treated mice, while mice administered dying PIT-treated cells exhibited tumor rejection and 90% tumor-free survival (FIGS. 10D-10E). These results indicate that vaccination with dying tumor cells, but not dead tumor cells, confers tumor immunity. These results also support that ICD, caused by PIT, stimulates the immune system to create anticancer immunity, and cells undergoing PIT-induced ICD can be used to vaccinate subjects to prevent future cancer growth.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a tumor or a lesion comprising:
administering to a first subject a composition comprising an illuminated sample, wherein the illuminated sample comprises tumor cells that have been treated ex vivo with photoimmunotherapy, wherein the photoimmunotherapy comprises:
i) contacting a sample of tumor cells ex vivo with a conjugate comprising a silicon phthalocyanine dye linked to a targeting molecule; and
ii) after contacting with the conjugate, illuminating the sample at a wavelength of at or about 600 nm to at or about 850 nm to obtain the illuminated sample,
wherein the tumor cells or a portion thereof within the illuminated sample exhibit one or more markers of immunogenic cell death (ICD).

2. A method of treating a tumor or a lesion comprising:
contacting a sample of tumor cells ex vivo with a conjugate comprising a silicon phthalocyanine dye linked to a targeting molecule;
after contacting with the conjugate, illuminating the sample at a wavelength of at or about 600 nm to at or about 850 nm to obtain an illuminated sample; and
administering a composition comprising the illuminated sample to a first subject,
wherein the tumor cells or a portion thereof within the illuminated sample exhibit one or more markers of immunogenic cell death (ICD).

3. The method of claim 1 or 2, wherein the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

4. The method of any of claims 1-3, wherein the composition exhibits less than 100% cell death prior to administration.

5. The method of any of claims 1-4, wherein the composition exhibits between about 30% and about 70% cell death prior to administration.

6. The method of any of claims 1-5, wherein the sample comprises tumor cells derived or obtained from the first subject.

7. The method of any of claims 1-5, wherein the sample comprises tumor cells derived or obtained from a second subject.

8. The method of any of claims 1-7, wherein the sample comprises tumor cells that have been grown or cultivated in vitro prior to contacting with the conjugate.

9. The method of claim 8, wherein the sample comprises tumor cells that have been grown or cultivated into an organoid prior to contacting with the conjugate.

10. The method of any of claims 1-9, wherein the composition is treated to prevent cell growth or cell expansion prior to administration to the first subject.

11. The method of claim 10, wherein the composition is treated by irradiation to prevent cell growth or cell expansion.

12. The method of claim 11, wherein the irradiation comprises gamma irradiation.

13. The method of any one of claims 1-12, wherein the first subject has been diagnosed as having, or is suspected of having, a type of cancer.

14. The method of claim 13, wherein the sample comprises tumor cells that are derived from the same or similar type of cancer.

15. The method of claim 13 or 14, wherein the type of cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, cancer of the blood, leukemia, lymphoma, and multiple myeloma, and any combination thereof.

16. The method of any of claims 1-15, wherein administering the composition results in a stimulation of an anti-cancer immune response in the first subject.

17. The method of any of claims 1-16, wherein administering the composition results in a reduction of growth, a reduction in size, a reduction in volume, or elimination of a tumor, a lesion or a metastasis in the first subject.

18. The method of any of claims 1-17, wherein the composition is administered by injection or by infusion to the first subject.

19. The method of any of claims 1-17, wherein the composition is administered by implantation into the first subject.

20. The method of any of claims 1-19, wherein the targeting molecule comprises an antibody or an antigen binding fragment thereof.

21. The method of any of claims 1-20, wherein the targeting molecule binds to a cell surface molecule.

22. The method of claim 21, wherein the cell surface molecule is present on a first tumor cell or a first cell in the tumor microenvironment, optionally wherein the sample comprises the first tumor cell or the first cell in the tumor microenvironment.

23. The method of claim 21 or 22, wherein the cell surface molecule is selected from the group consisting of HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, a ganglioside, VEGF receptor (VEGFR), VEGFR2, VEGF-A, integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, SK-1antigen, Bcr-abl, RET, MET, TRKB, TIE2, ALK, ROS, EML4-ALK, ROS1, BRAFV600E, SRC, c-KIT, PDGFR, mTOR, TSC1, TSC2, BTK, KIT, BRCA, CDK 4/6, JAK1, JAK2, BRAF, FLT-3, MEK1, MEK2, and SMO.

24. The method of any of claims 1-23, wherein the targeting molecule is selected from the group consisting of cetuximab, panitumumab, zalutumumab, nimotuzumab, Tositumomab (Bexxar®), Rituximab (Rituxan, MabThera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002, and MNRP1685A, and any antigen-binding fragment thereof.

25. The method of any of claims 1-24, wherein the silicon phthalocyanine dye is IR700.

26. The method of any of claims 1-25, further comprising administering a second treatment to the first subject, wherein the second treatment comprises:
administering to the first subject a second conjugate comprising a second phthalocyanine dye linked to a second targeting molecule, and
after administering the second conjugate, illuminating a tumor or a lesion in the first subject at a wavelength of at or about 600 nm to at or about 850 nm and at a dose of from at or about 25 J/cm² to at or about 400 J/cm² or from at or about 2 J/cm fiber length to at or about 500 J/cm fiber length.

27. The method of claim 26, wherein the second treatment is administered subsequent to administering the composition to the first subject.

28. The method of claim 26, wherein the second treatment is administered prior to administering the composition to the first subject.

29. The method of claim 26, wherein the second treatment is administered prior to administering of the composition to the first subject.

30. The method of any of claims 1-29, wherein the composition is administered in combination with an immune modulatory agent.

31. The method of claim 30, wherein the immune modulatory agent is administered prior to, concurrent with and/or subsequent to the composition.

32. The method of claim 30 or 31, wherein the immune modulatory agent comprises an adjuvant, an immune checkpoint inhibitor, a cytokine or any combination thereof.

33. The method of any of claims 1-32, wherein the tumor cells are obtained from multiple tumor sources.

34. The method of any of claims 1-33, wherein the sample comprises tumor cells from a tumor or a lesion associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.

35. A pharmaceutical composition, comprising photoimmunotherapy-treated tumor cells formulated with at least one pharmaceutically acceptable excipient.

36. The pharmaceutical composition of claim 35, wherein the tumor cells or a portion thereof in the pharmaceutical composition exhibit one or more markers of immunogenic cell death (ICD).

37. The pharmaceutical composition of claim 35 or 36, wherein the one or more markers of ICD is selected from the group consisting of annexin, adenosine triphosphate release, interferon α release, interferon β release, release of a high mobility group I protein, cell surface expression of HSP70, cell surface expression of HSP90, and cell surface expression of calreticulin.

38. The pharmaceutical composition of any of claims 35-37, wherein the tumor cells within the pharmaceutical composition exhibit less than 100% cell death.

39. The pharmaceutical composition of any of claims 35-38, wherein the tumor cells within the pharmaceutical composition exhibit between about 30% and about 70% cell death.

40. The pharmaceutical composition of any of claims 35-39, comprising tumor cells derived from a single subject.

41. The pharmaceutical composition of any of claims 35-39, comprising tumor cells derived from more than one subject.

42. The pharmaceutical composition of any of claims 35-41, comprising tumor cells derived from a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, a blood cancer, leukemia, lymphoma, and multiple myeloma, and any combination thereof.
